(19) United States
(12) Patent Application Publication
(10) Pub. No.: US 2024/0203552 A1
(43) Pub. Date: Jun. 20, 2024

(54) VIDEO SURGICAL REPORT GENERATION

(71) Applicant: Stryker Corporation, Kalamazoo, MI (US)

(72) Inventors: Jose GEORGE, Kollam (IN); Sanskruti MASKE, Gondia (IN)

(73) Assignee: Stryker Corporation, Kalamazoo, MI (US)

(21) Appl. No.: 18/539,090

(22) Filed: Dec. 13, 2023

Related U.S. Application Data
(60) Provisional application No. 63/387,929, filed on Dec. 16, 2022.

(51) Int. Cl. G16H 15/00; G06V 10/74; G06V 20/40; G10L 13/033

(52) U.S. Cl. CPC G16H 15/00; G06V 10/761; G06V 20/41; G06V 20/46; G10L 13/033; G06V 2201/03

(57) ABSTRACT

Disclosed herein are methods for generating a video surgical report using a machine learning pipeline. The machine learning pipeline may include one or more machine learning models, each of which may support a particular aspect of a video surgical report generation process. For example, one or more images of a surgical procedure may be obtained. Using one or more machine learning models, a set of images from the one or more images may be selected based on the surgical procedure. A video surgical report may be generated for the surgical procedure, which may include at least some of the set of images. The machine learning pipeline can offload work typically performed by a user (e.g., surgeon, medical staff, etc.) to create the video surgical report, thereby saving significant time and/or resources.

### CROSS-REFERENCE TO RELATED APPLICATIONS

This application claims the benefit of U.S. Provisional Application No. 63/387,929, filed Dec. 16, 2022, the entire contents of which are hereby incorporated by reference herein.

### FIELD

The present application generally relates to generating video surgical reports and, in particular, using machine learning to generate content for video surgical reports.

### BACKGROUND

A surgical report is often used to explain to a patient what occurred during a surgical procedure. These surgical procedures include a description of important events that occurred during the surgery. Generating these reports can be a time consuming and inaccurate process. This is because the generation of these reports relies on the surgeon recalling what events occurred during the surgery, after surgery (i.e., during the report generation process). Since the generation of this report is not contemporaneous with the procedure, the surgeon may not identify all of the events from the surgery and/or may not recall all of the circumstances surrounding those events which have been identified. Further, the generation of these reports takes up valuable time of the surgeon.

Therefore, it would be beneficial to have systems, methods, and programming that produce improved surgical reports and processes for creating surgical reports.

### SUMMARY

Described are systems, methods, and programming for a machine learning pipeline used to generate a video surgical report. The systems, methods, and programming can be configured to automatically generate video content for the video surgical report. The machine learning pipeline may include one or more machine learning models, each supporting a particular aspect of a video surgical report generation process. The machine learning pipeline can offload much of the work typically performed by the surgeon to create the video surgical report, thereby saving significant time. Additionally, the machine learning pipeline may intelligently curate content for the video surgical report. This curated content may be selected to highlight key surgical events from the surgical procedure. The machine learning models may also generate text, audio, video, or other media for the video surgical report.

The machine learning pipeline may include one or more machine learning models relating to various portions of the video surgical report generation process. For example, the machine learning models may include one or more models relating to information distillation, information extraction, language generation, language translation, audio generation, video generation, etc. Some or all of the machine learning models may access images from a surgical video feed, such as images captured by an endoscope. These images may be used to create the video surgical report describing the surgical procedure.

During an information distillation stage of the video surgical report generation process, one or more machine learning models may analyze the images to determine a surgical phase, surgical activities performed during the surgical phase, or other information. The determined surgical phase and/or surgical activities may be provided to an information extraction stage of the video surgical report generation process. During the information extraction stage, the images may be analyzed using one or more machine learning models to determine whether the images depict any key surgical events and/or anatomical structures. The key surgical events refer to rare and/or unexpected events. Additionally, the machine learning models may determine anatomical structure measurement information associated with the key surgical events and/or the anatomical structures. The anatomical structure measurement information may include measurements describing physical characteristics of anatomical structures determined to be depicted by the images (e.g., shape, size, volume, etc.). The machine learning models may also select a set of images from the images analyzed based on whether the images depict a key surgical event, anatomical structure, or another aspect of the surgical procedure.

The set of images, key surgical events, information describing anatomical structures, anatomical structure quantification information, or other information may be provided to a language generation stage of the video surgical report generation process. During the language generation stage, one or more machine learning models may generate a description for the set of images to be included in the video surgical report. The description may be created based on the key surgical events, anatomical structures, anatomical structure quantification information, or other information. If necessary, the description may be translated to one or more additional languages during a language translation stage. Additionally, the description may be generated in different styles depending on the target audience. For example, one vocabulary may be used to form descriptions targeted to a first audience (e.g., for training individuals) and another vocabulary may be used to form descriptions targeted to a second audience (e.g., for auditing medical procedures). Different language translation models may be trained to translate the description from one language to the additional languages. During an audio generation stage, audio may be generated based on the description, the set of images, and/or other information identified during the video surgical report generation process. The audio may be generated such that it has sound characteristics similar to the vocal characteristics of the surgeon (or another medical professional).

Using the set of images, the generated description, the generated audio, and/or other information, a video surgical report may be generated during a video generation stage. The video surgical report may incorporate some or all of the images, text from the generated description, and/or graphics to help describe various aspects of the surgical procedure. The video surgical report may include a virtual character (e.g., an avatar) programmed to speak the generated audio. For example, data programming facial movements and expressions of the virtual character may be included in the video surgical report such that the virtual character appears to utter the generated text. The video surgical report may be created with minimal manual input from the surgeon, thereby reducing the amount of time the surgeon needs to devote to creating a surgical report.

According to some examples, a method includes obtaining one or more images of a surgical procedure; determining, using one or more machine learning models, a set of images from the one or more images based on the surgical procedure; and generating a video surgical report for the surgical procedure comprising at least some of the set of images. The set of images can comprise fewer images than the obtained one or more images of the surgical procedure. Therefore, the set of images can form a compressed representation of the obtained one or more images of the surgical procedure.

In any of the examples, generating the video surgical report can include generating, using the one or more machine learning models, text describing the at least some of the set of images, wherein the video surgical report comprises at least some of the text corresponding to the at least some of the set of images.

In any of the examples, generating the video surgical report can include generating a virtual character programmed to output audio associated with the at least some of the set of images, the video surgical report comprising the virtual character.

In any of the examples, determining the set of images can include determining, using the one or more machine learning models, at least one of: a phase of the surgical procedure, a surgical activity being performed during the phase, or information related to the set of images.

In any of the examples, determining the set of images can include selecting the set of images from the one or more images based on the at least one of the phase, the surgical activity, or the information related to the set of images.

In any of the examples, the method can further include training the one or more machine learning models to analyze content depicted by the set of images to determine the at least one of the phase, the surgical activity, or the information related to the set of images.

In any of the examples, the one or more images can include a first image and a second image captured during a same phase of the surgical procedure, determining the set of images can include computing, using the one or more machine learning models, a first classification score and a second classification score respectively associated with the first image and the second image; and adding at least one of the first image or the second image to the set of images based on the first classification score and the second classification score.

In any of the examples, the method can further include identifying at least one image from the one or more images based on preoperative information related to the surgical procedure, wherein the set of images comprises the at least one image.

In any of the examples, obtaining the one or more images can include identifying, using the one or more machine learning models, a subset of frames from a video of the surgical procedure that depict one or more objects associated with the surgical procedure, wherein the one or more images comprise the subset of frames.

In any of the examples, the method can further include detecting, using the one or more machine learning models, one or more objects associated with the surgical procedure within the one or more images, wherein the at least some of the set of images are selected based on the one or more objects.

In any of the examples, the method can further include receiving preoperative information related to at least one of the surgical procedure or a patient associated with the surgical procedure; and generating content to be included in the video surgical report based on the received preoperative information.

In any of the examples, the method can further include generating, using the one or more machine learning models, text associated with the set of images; and associating one or more portions of the text with the set of images.

In any of the examples, the method can further include generating, using the one or more machine learning models, first text associated with the set of images, the first text being in a first language; and transforming, using the one or more machine learning models, the first text into second text, the second text being in a second language, the video surgical report comprising at least some of the second text corresponding to the at least some of the set of images.

In any of the examples, generating the video surgical report can include generating, using the one or more machine learning models, text for the at least some of the set of images; and generating, using the one or more machine learning models, audio based on the text, the video surgical report comprising the audio. Hence, audio information representative of the at least some of the set of images can be generated, based on the at least some of the set of images.

In any of the examples, the method can further include receiving, using one or more audio sensors, audio captured during the surgical procedure, wherein the video surgical report comprises at least some of the audio corresponding to the at least some of the set of images.

In any of the examples, generating the video surgical report can include obtaining pre-generated text associated with content depicted by the at least some of the set of images; obtaining user-provided text of audio captured during the surgical procedure; and generating text for the video surgical report based on the pre-generated text and the user-provided text.

In any of the examples, generating the video surgical report can include generating audio based on data stored in an audio profile of a user, the data comprising at least one of a pitch, a timbre, a loudness, or a modulation associated with the user.

In any of the examples, obtaining the one or more images can include accessing video captured during the surgical procedure; and extracting at least one video snippet from the video based on the surgical procedure, wherein the one or more images comprise the at least one video snippet.

In any of the examples, the method can further include obtaining one or more additional images captured subsequent to the surgical procedure; and generating an updated video surgical report comprising at least some of the one or more additional images.

In any of the examples, generating the video surgical report can include adding one or more additional images to the video surgical report based on a similarity between content depicted by the one or more additional images and content depicted by at least one of the set of images, wherein the one or more additional images are captured prior to the surgical procedure.

According to some examples, a non-transitory computer-readable medium stores computer program instructions that, when executed by one or more processors, effectuate the method of any one of any of the examples.

According to some examples, a computer program product comprises software code portions including computer program instructions that, when executed by one or more processors, effectuate the method of any one of any of the examples.

According to some examples, a system includes: memory storing computer program instructions; and one or more processors configured to execute the computer program instructions to cause the one or more processors to perform the method of any of the examples.

According to some examples, a medical device, includes: one or more processors programmed to perform the method of any of the examples.

In any of the examples, the medical device can further include: an image sensor configured to capture the one or more images of the surgical procedure.

It will be appreciated that any of the variations, aspects, features, and options described in view of the methods apply equally to the systems and devices, and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1A illustrates an example medical environment, according to some aspects.

FIG. 1B illustrates an example system for generating a video surgical report, according to some aspects.

FIG. 2 illustrates an example machine learning pipeline used to generate a video surgical report, according to some aspects.

FIG. 3 illustrates an example model database including machine learning models used within a machine learning pipeline, according to some aspects.

FIG. 4 illustrates an example system for training and implementing a machine learning model to detect phases of a surgical procedure, according to some aspects.

FIG. 5 illustrates an example system for training and implementing a machine learning model to detect key surgical events and extract images/videos associated with the key surgical events, according to some aspects.

FIG. 6 illustrates an example system for training and implementing a machine learning model to generate text describing the key surgical events, according to some aspects.

FIG. 7 illustrates an example system for training and implementing a machine learning model to translate text describing key surgical events from a first language to a second language, according to some aspects.

FIG. 8 illustrated an example system for training and implementing a machine learning model to generate audio based on text describing key surgical events, according to some aspects.

FIG. 9 illustrates an example system for training and implementing a machine learning model to generate a video surgical report based on generated audio, generated text, extracted images/video, or other information, according to some aspects.

FIG. 10 illustrates an example video surgical report, according to some aspects.

FIG. 11 illustrates an example flowchart of a method for generating a video surgical report, according to some aspects.

FIG. 12 illustrates an example computing system used for performing any of the techniques described herein, according to some aspects.

### DETAILED DESCRIPTION

Reference will now be made in detail to implementations and various aspects and variations of systems and methods described herein. Although several example variations of the systems and methods are described herein, other variations of the systems and methods may include aspects of the systems and methods described herein combined in any suitable manner having combinations of all or some of the aspects described.

Described are systems, methods, and programming for generating a video surgical report and a machine learning pipeline used for generating the report. The video surgical report may include images, video, text, virtual characters, or other information describing a surgical procedure. Various portions of the surgical report may utilize different information. For example, a set of images for the surgical report may be selected from one or more images of a surgical procedure based on key surgical events. These key surgical events may be identified based on distillation information, such as surgical phases, surgical activities, and/or information related to the selected images. As such, the video surgical report may comprise a compressed representation of the one or more images of the surgical procedure. As another example, audio for the surgical report may be generated based on text descriptions describing the key surgical events and/or the selected images. The text may be created based on the identified images and/or information extracted from those images (e.g., anatomical structures determined to be depicted by the images). The text, the audio, the images, or other information may be utilized to generate video for the surgical report.

The machine learning pipeline may include one or more machine learning models, each configured to perform certain tasks associated with the video surgical report generation process. For example, the machine learning pipeline may include an information distillation stage of the machine learning pipeline (e.g., a surgical phase detection model, a surgical activity detection model, etc.), one or more machine learning models associated with an information extraction stage of the machine learning pipeline (e.g., a surgical event detection model, a surgical anatomy identification model, a surgical anatomy measurement model, etc.), one or more machine learning models associated with a language generation and/or language translation stage of the machine learning pipeline (e.g., a synthetic description generation model, a synthetic description translation model), one or more machine learning models associated with an audio generation stage of the machine learning pipeline (e.g., a synthetic audio generation model), one or more machine learning models associated with a video generation stage of the machine learning pipeline (e.g., a synthetic video generation model), or other models. Some or all of the machine learning models may access a surgical feed (e.g., images and/or videos) of a surgical procedure. The images and/or videos may include those captured by a medical device, such as an endoscope. Pre-processing may be performed to the surgical feed prior to being analyzed by the machine learning models. For example, a video of the surgical procedure captured by an image sensor of a medical device can be parsed into frames (e.g., a sequence of images forming a video). As another example, preoperative information (such as preoperative medical exam results, preoperative medical images, or other preoperative information associated with the surgical procedure, the surgeon to be performing the surgical procedure, the patient to whom the surgical procedure is to be performed, etc.) may be obtained and provided to the machine learning pipeline.

The machine learning models may obtain images (e.g., video frames) of the medical procedure, such as those captured by a medical device (e.g., an endoscope). These images may be provided to some or all of the aforementioned machine learning models, at least a portion of which may generate outputs capable of being fed as input to one or more downstream models. The result of the machine learning pipeline may be a video surgical report that clearly and concisely explains the surgical procedure that was performed using images, video, text, audio, virtual characters, and/or other information. The video surgical report produced by the machine learning pipeline may minimize what, if any, input is needed from the surgeon. Thus, the complex and time-consuming process of creating a surgical report may be offloaded from the surgeon.

It should be noted that although some aspects are described herein with respect to machine learning models, other prediction models (e.g., statistical models or other analytics models) may be used in lieu of or in addition to the machine learning models described herein. For example, a statistical model may replace a machine-learning model and a non-statistical model may replace a non-machine-learning model.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. Suitable processors include central processing units (CPUs), graphical processing units (GPUs), field-programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

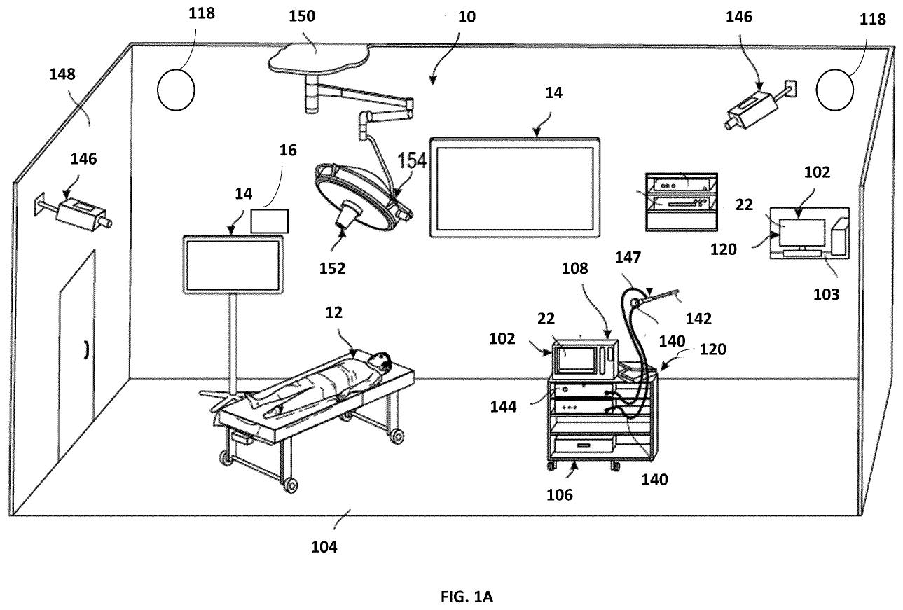

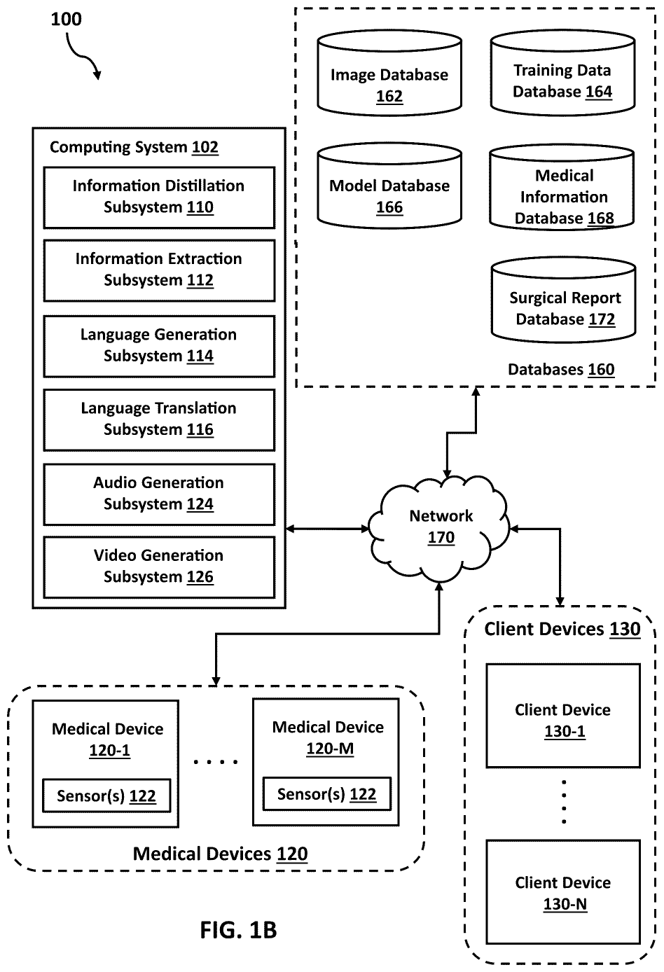

FIG. 1A illustrates an example medical environment 10, according to some aspects. Medical environment 10 may represent a surgical suite, operating room, or other care environment where a medical procedure may be performed. Furthermore, activities may be performed within medical environment 10 that occur prior to or after a medical procedure has been performed. For example, activities that may occur prior to the medical procedure beginning may include, but are not limited to, setting up medical equipment, preparing mixtures, cleaning surfaces/objects, wheeling a patient into medical environment 10, placing a patient on a surgical table, and/or dispensing anesthetics. As another example, activities that may occur after the medical procedure has ended may include moving a patient to a stretcher or wheelchair, wheeling the patient out of medical environment 10, cleaning surfaces/objects, or moving medical devices to different locations.

Medical environment 10 may include devices used to prepare for and/or perform a medical procedure to a patient 12. These devices may also be used after the medical procedure. Such devices may include one or more sensors, one or more medical devices, one or more display devices, one or more light sources, one or more computing devices, or other components. For example, at least one medical device 120 may be located within medical environment 10. Medical device 120 may be used to assist medical staff while performing a medical procedure (e.g., surgery). Medical device 120 may also be used to document events and information from the medical procedure. For example, medical device 120 may be used to input or receive patient information (e.g., to/from electronic medical records (EMRs), electronic health records (EHRs), hospital information system (HIS), communicated in real-time from another system, etc.). The received patient information may be saved onto medical device 120. Alternatively or additionally, the patient information may be displayed using medical device 120. In some aspects, medical device 120 may be used to record patient information. For example, medical device 120 may be used to store the patient information or images in an EMR, EHR, HIS, or other databases.

Medical device 120 may be capable of obtaining, measuring, detecting, and/or saving information related to patient 12. Medical device 120 may or may not be coupled to a network that includes records of patient 12, for example, an EMR, EHR, or HIS. Medical device 120 may include or be integrated with a computing system 102 (e.g., a desktop computer, a laptop computer, a tablet device, etc.) having an application server. For example, medical device 120 may include processors or other hardware components that enable data to be captured, stored, saved, and/or transmitted to other devices. Computing system 102 can have a motherboard that includes one or more processors or other similar control devices as well as one or more memory devices. The processors may control the overall operation of computing system 102 and can include hardwired circuitry, programmable circuitry that executes software, or a combination thereof. The processors may, for example, execute software stored in the memory device. The processors may include, for example, one or more general- or special-purpose programmable microprocessors and/or microcontrollers, graphics processing unit (GPU), tensor processing unit (TPU), application specific integrated circuits (ASICs), programmable logic devices (PLDs), programmable gate arrays (PGAs), or the like. The memory devices may include any combination of one or more random access memories (RAMs), read-only memories (ROMs) (which may be programmable), flash memory, and/or other similar storage devices. Patient information may be input into computing system 102 (e.g., making an operative note during the medical or surgical procedure on patient 12 in medical environment 10) and/or computing system 102 can transmit the patient information to another medical device 120 (via either a wired connection or wirelessly).

Computing system 102 can be positioned in medical environment 10 on a table (stationary or portable), a floor 104, a portable cart 106, an equipment boom, and/or a shelving 103. FIG. 1A illustrates two forms of computing system 102: a first computing system in the form of a desktop computer on shelving 103 and a second computing system incorporated into an imaging system 108 on portable cart 106. Further, examples of the disclosure may include any number of computer systems.

In some aspects, medical environment 10 may be an integrated suite used for minimally invasive surgery (MIS) or fully invasive procedures. Video components, audio components, and associated routing may be located throughout medical environment 10. For example, monitor 14 may present video and speakers 118 may output audio. The components may be located on or within the walls, ceilings, or floors of medical environment 10. For example, room cameras 146 may be mounted to walls 148 or a ceiling 150. Wires, cables, and hoses can be routed through suspensions, equipment booms, and/or interstitial space. The wires, cables, and/or hoses in medical environment 10 may be capable of connecting to mobile equipment, such as portable cart 106, C arms, microscopes, etc., to communicate routing audio, video, and data information.

Imaging system 108 may be configured to capture images and/or video, and may route audio, video, and other data (e.g., device control data) throughout medical environment 10. Imaging system 108 and/or associated router(s) may route the information between devices within or proximate to medical environment 10. In some aspects, imaging system 108 and/or associated router(s) (not shown) may be located external to medical environment 10 (e.g., in a room outside of an operating room), such as in a closet. As an example, the closet may be located within a predefined distance of medical environment 10 (e.g., within 325 feet, or 100 meters). In some aspects, imaging system 108 and/or the associated router(s) may be located in a cabinet inside or adjacent to medical environment 10.

The captured images and/or videos may be displayed via one or more display devices. For example, images captured by imaging system 108 may be displayed using monitor 14. Imaging system 108, alone or in combination with one or more audio sensors, may also be capable of recording audio, outputting audio, or a combination thereof. In some aspects, patient information can be input into imaging system 108 and added to the images and videos recorded and/or displayed. Imaging system 108 can include internal storage (e.g., a hard drive, a solid state drive, etc.) for storing the captured images and videos. Imaging system 108 can also display any captured or saved images (e.g., from the internal hard drive). For example, imaging system 108 may cause monitor 14 to display a saved image. As another example, imaging system 108 may display a saved video using a touchscreen monitor 22. Touchscreen monitor 22 and/or monitor 14 may be coupled to imaging system 108 via a wired connection and/or wirelessly. It is contemplated that imaging system 108 could obtain or create images of patient 12 during a medical or surgical procedure from a variety of sources (e.g., from video cameras, video cassette recorders, X-ray scanners (which convert X-ray films to digital files), digital X-ray acquisition apparatus, fluoroscopes, computed tomography (CT) scanners, magnetic resonance imaging (MRI) scanners, ultrasound scanners, charge-coupled (CCD) devices, and other types of scanners (handheld or otherwise)). If coupled to a network, imaging system 108 can also communicate with a picture archiving and communication system (PACS), as is well known to those skilled in the art, to save images and videos in the PACS and to retrieve images and videos from the PACS. Imaging system 108 can couple to and/or integrate with, e.g., an electronic medical records database (e.g., EMR) and/or a media asset management database.

Touchscreen monitor 22 and/or monitor 14 may display images and videos captured live by imaging system 108. Imaging system 108 may include at least one image sensor, for example, disposed within camera head 140. Camera head 140 may be configured to capture an image or a sequence of images (e.g., video frames) of patient 12. Camera head 140 can be a hand-held device, such as an open-field camera or an endoscopic camera. For example, imaging system 108 may be coupled to an endoscope 142, which may include or be coupled to, camera head 140. Imaging system 108 may communicate with a camera control unit 144 via a fiber optic cable 147, which may communicate with imaging system 108 (e.g., via a wired or wireless connection).

Room cameras 146 may also be configured to capture an image or a sequence of images (e.g., video frames) of medical environment 10. The captured image(s) may be displayed using touchscreen monitor 22 and/or monitor 14. In addition to room cameras 146, a camera 152 may be disposed on a surgical light 154 within medical environment 10. Camera 152 may be configured to capture an image or a sequence of images of medical environment 10 and/or patient 12. Images captured by camera head 140, room cameras 146, and/or camera 152 may be routed to imaging system 108, which may then be displayed using touchscreen monitor 22, monitor 14, another display device, or a combination thereof. Additionally, the images captured by camera head 140, room cameras 146, and/or camera 152 may be provided to a database for storage (e.g., an EMR).

Room cameras 146, camera 152, and/or camera head 140 of endoscope 142 (or another camera of imaging system 108) may include at least one solid state image sensor. For example, the image sensor of room cameras 146, camera 152, and/or camera head 140 may include a charge coupled device (CCD), a complementary metal-oxide semiconductor (CMOS) sensor, a charge-injection device (CID), or another suitable sensor technology. The image sensor of room cameras 146, camera 152, and/or camera head 140 may include a single image sensor. The single image sensor may be a grayscale image sensor or a color image sensor having an RGB color filter array deposited on its pixels. The image sensor of room cameras 146, camera 152, and/or camera head 140 may alternatively include three sensors: one sensor for detecting red light, one sensor for detecting green light, and one sensor for detecting blue light.

The medical procedure in which the images may be captured using room cameras 146, camera 152, and/or camera head 140 may be an exploratory procedure, a diagnostic procedure, a study, a surgical procedure, a non-surgical procedure, an invasive procedure, or a non-invasive procedure. As mentioned above, camera head 140 may be an endoscopic camera (e.g., coupled to endoscope 142). It is to be understood that the term endoscopic (and endoscopy in general) is not intended to be limiting, and rather camera head 140 may be configured to capture medical images from various scope-based procedures including but not limited to arthroscopy, ureteroscopy, laparoscopy, colonoscopy, bronchoscopy, etc.

Speakers 118 may be positioned within medical environment 10 to provide sounds, such as music, audible information, and/or alerts, that can be played within the medical environment during the medical procedure. For example, speaker(s) 118 may be installed on ceiling 150 and/or positioned on a bookshelf, on a station, etc.

One or more microphones 16 may sample audio signals within medical environment 10. The sampled audio signals may comprise the sounds played by speakers 118, noises from equipment within medical environment 10, and/or human speech (e.g., voice commands to control one or more medical devices or verbal information conveyed for documentation purposes). Microphone(s) 16 may be located within a speaker (e.g., a smart speaker) attached to monitor 14, as shown in FIG. 1A, and/or within the housing of touchscreen monitor 22. Microphones 16 may communicate via a wired and/or a wireless connection with imaging system 108 and/or computing system 102. Microphones 16 may provide, record, and/or process the sampled audio signals. Microphones 16 may determine and record the surgeon's speech for documentation purposes (e.g., recording verbal information for educational purposes, making room calls, sending real-time information to pathologists, etc.). Additionally or alternatively, microphones 16 may be capable of processing the sampled audio signals, including recognizing the voice commands received from the surgeon (or a medical professional). The captured audio signal may also be processed using computing system 102.

FIG. 1B is a diagram illustrating an example system 100, according to some aspects. System 100 may include computing system 102, medical devices 120 (e.g., medical device 120-1 to medical device 120-M), client devices 130 (e.g., client device 130-1 to client device 130-N), databases 160 (e.g., image database 162, training data database 164, model database 166, medical information database 168, surgical report database 172), or other components. Components of system 100 may communicate with one another using network 170 (e.g., the Internet).

Medical devices 120 may include one or more sensors 122, such as an image sensor, an audio sensor, a motion sensor, or other types of sensors. Image sensors may be configured to capture one or more images, one or more videos, audio, or other data relating to a medical procedure. As an example, with reference to FIG. 1A, medical device 120 may correspond to endoscope 142. Endoscope 142 may include a camera head 140 having one or more image sensors. Medical device 120 may be used to obtain or create images of patient 12 during a medical procedure from a variety of sources (e.g., from video cameras, video cassette recorders, X-ray scanners (which can convert X-ray films to digital files), digital X-ray acquisition apparatus, fluoroscopes, computed tomography (CT) scanners, magnetic resonance imaging (MRI) scanners, ultrasound scanners, charge-coupled (CCD) devices, and other types of scanners (handheld or otherwise)). For example, medical device 120 may capture images and/or videos depicting an anatomical structure of patient 12, as seen with respect to FIG. 1A. As another example, medical device 120 may be a medical imaging device (e.g., MRI machines, CT machines, X-Ray machines, etc.). As yet another example, medical device 120 may be a biometric data capture device (e.g., a blood pressure device, pulse-ox device, etc.).

Client devices 130-1 to 130-N may be capable of communicating with one or more components of system 100 via a wired and/or wireless connection (e.g., network 170). Client devices 130 may interface with various components of system 100 to cause one or more actions to be performed. For example, client devices 130 may represent one or more devices used to display images and videos to a user (e.g., a surgeon). Examples of client devices 130 may include, but are not limited to, desktop computers, servers, mobile computers, smart devices, wearable devices, cloud computing platforms, display devices, mobile terminals, fixed terminals, or other client devices. Each client device 130-1 to 130-N of client devices 130 may include one or more processors, memory, communications components, display components, audio capture/output devices, imaging components, other components, and/or combinations thereof.

Computing system 102 may include one or more subsystems, such as an information distillation subsystem 110, an information extraction subsystem 112, a language generation subsystem 114, a language translation subsystem 116, an audio generation subsystem 124, a video generation subsystem 126, or other subsystems. Some or all of information distillation subsystem 110, information extraction subsystem 112, language generation subsystem 114, language translation subsystem 116, audio generation subsystem 124, and video generation subsystem 126 may be implemented using one or more processors, memory, and interfaces. Distributed computing architectures and/or cloud-based computing architectures may alternatively or additionally be used to implement some or all of the functionalities associated with information distillation subsystem 110, information extraction subsystem 112, language generation subsystem 114, language translation subsystem 116, audio generation subsystem 124, and video generation subsystem 126.

It should be noted that, while one or more operations are described herein as being performed by particular components of computing system 102, those operations may be performed by other components of computing system 102 or other components of system 100. As an example, while one or more operations are described herein as being performed by components of computing system 102, those operations may alternatively be performed by one or more of medical devices 120 and/or client devices 130.

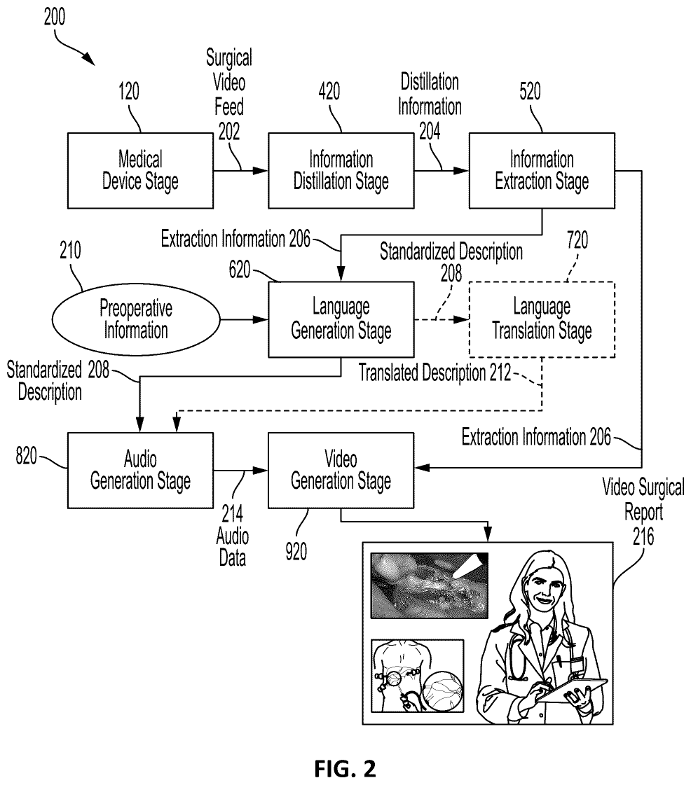

Information distillation subsystem 110, information extraction subsystem 112, language generation subsystem 114, language translation subsystem 116, audio generation subsystem 124, and/or video generation subsystem 126 may be configured to implement various portions of a video surgical report generation process. FIG. 2 illustrates an example video surgical report generation process 200. For example, with reference to FIG. 2, video surgical report generation process 200 may include one or more stages whereby information is determined, extracted, created, and/or formatted for inclusion within a surgical video report. Video surgical report generation process 200 may also be referred to as a machine learning pipeline, at least because video surgical report generation process 200 may include one or more machine learning models used to create the video surgical report.

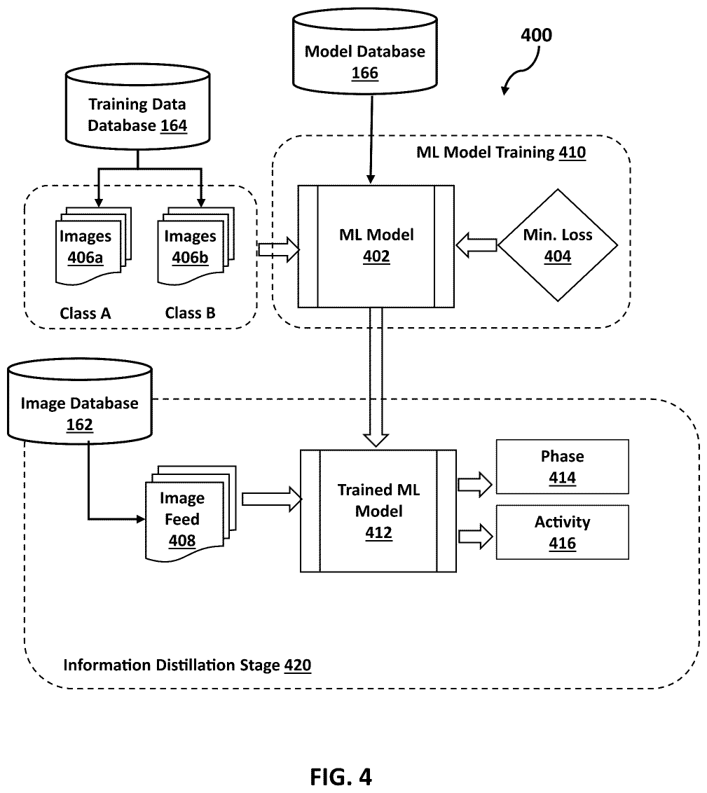

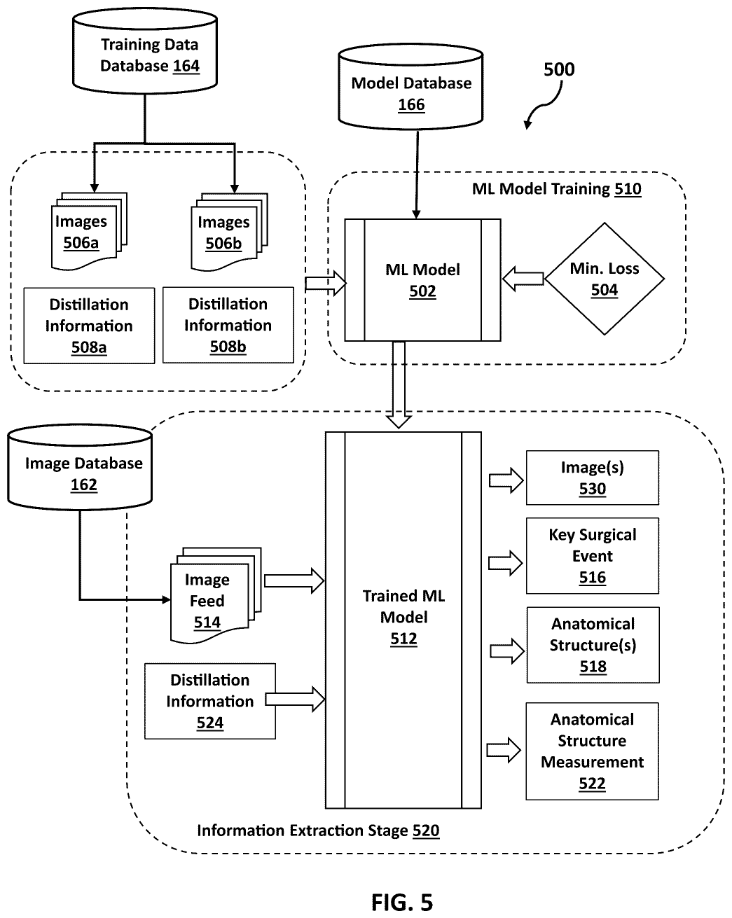

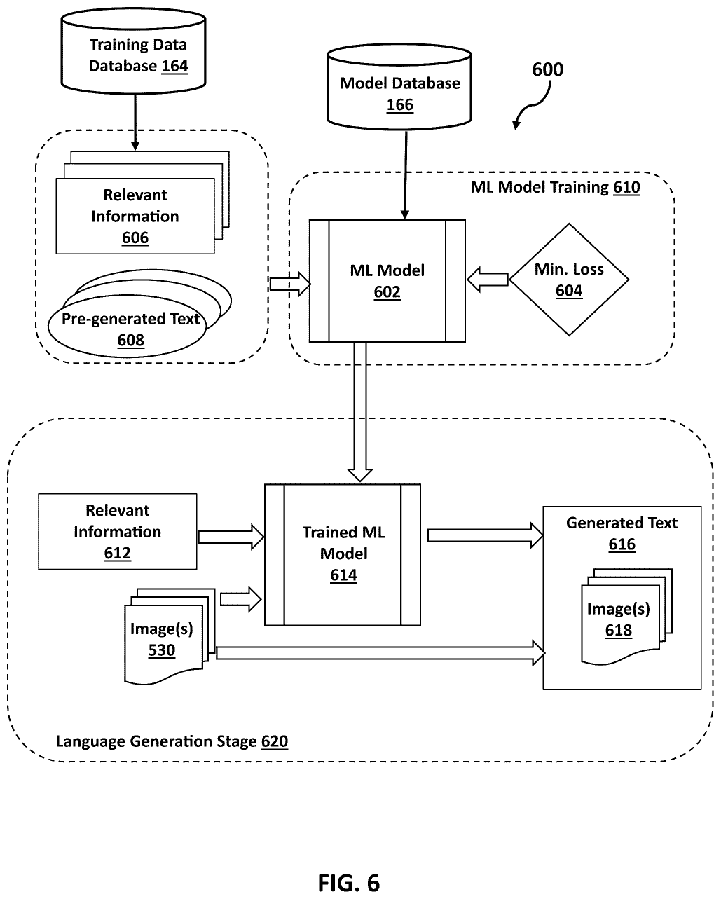

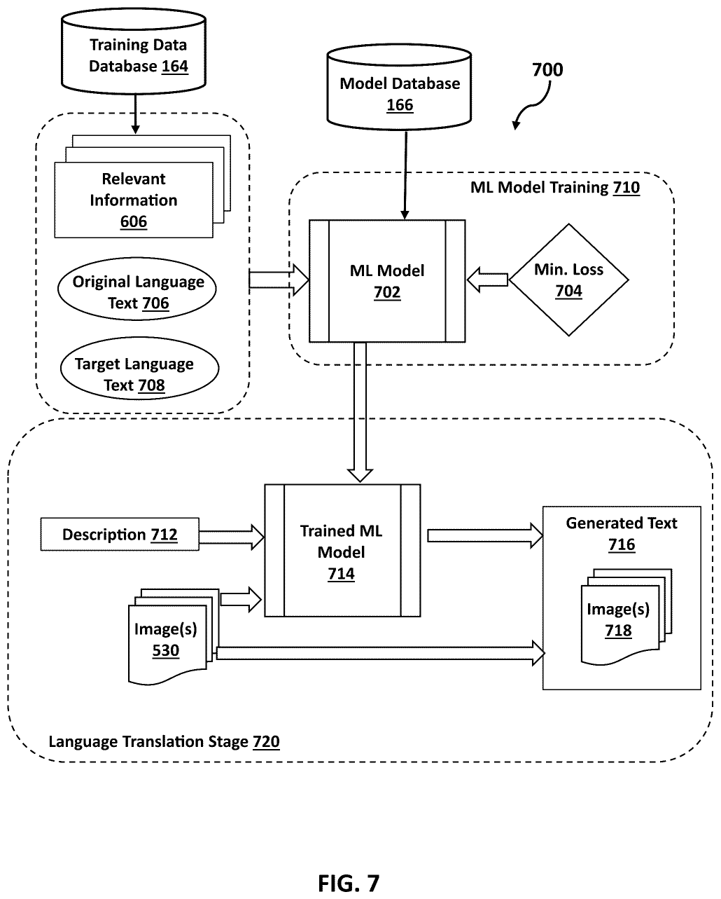

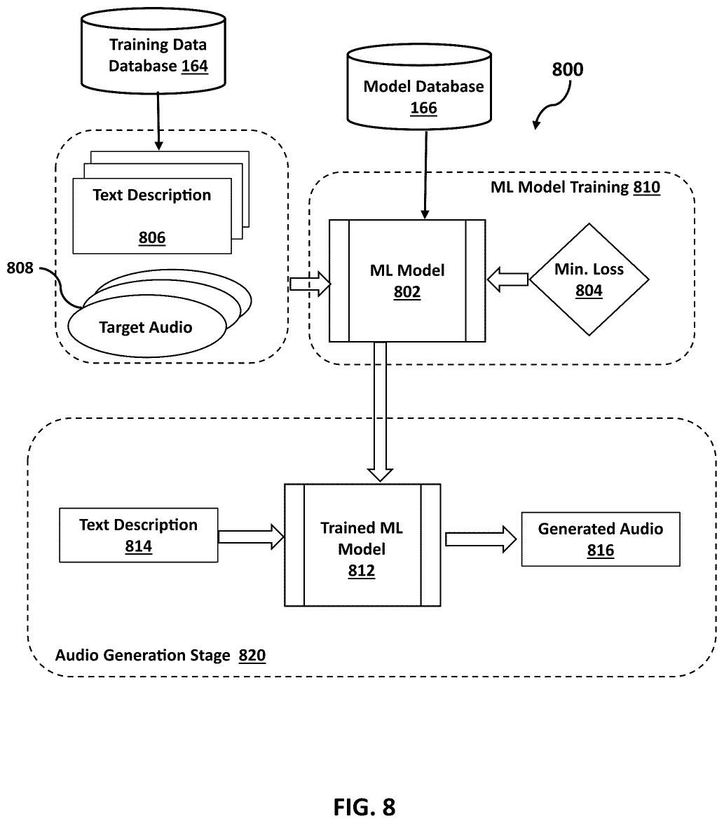

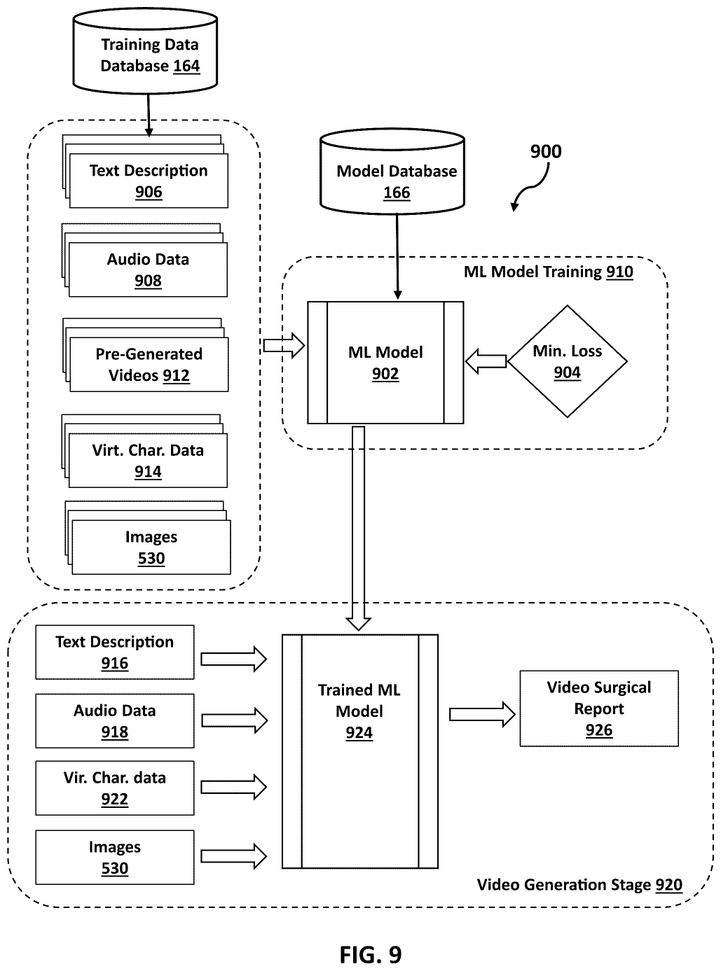

Video surgical report generation process 200 may include an information distillation stage 420 (shown in FIG. 4), an information extraction stage 520 (shown in FIG. 5), a language generation stage 620 (shown in FIG. 6), a language translation stage 720 (shown in FIG. 7), an audio generation stage 820 (shown in FIG. 8), a video generation stage 920 (shown in FIG. 9), or other stages. Persons of ordinary skill in the art will recognize that some or all of information distillation stage 420, information extraction stage 520, language generation stage 620, language translation stage 720, audio generation stage 820, and video generation stage 920 may also be combined or omitted. In video surgical report generation process 200 of FIG. 2, a medical device 120 (shown in FIG. 1B) may output surgical video feed 202. As mentioned previously, surgical video feed 202 may include one or more images, such as a sequence of images (e.g., frames). For example, surgical video feed 202 may be captured by camera head 140 of endoscope 142, as seen in FIG. 1A. Surgical video feed 202 may be stored in image database 162 (shown in FIG. 1B). For example, the sequence of images forming surgical video feed 202 may be stored in image database 162.

Surgical video feed 202 may be provided as input to information distillation stage 420, which may output distillation information 204. Examples of distillation information 204 may include a detected surgical phase and/or one or more surgical activities detected during the surgical phase.

Distillation information 204 may be provided as input to information extraction stage 520. At information extraction stage 520, extraction information 206 may be extracted from surgical video feed 202 based on distillation information 204. Extraction information 206 may include indications of any surgical events that were detected during the surgical procedure, as well as any anatomical structures that are identified. Extraction information 206 may also include details associated with the identified anatomical structures (e.g., length, volume, blood loss, etc.).

Extraction information 206 may be provided as input to language generation stage 620, which may output a standardized description 208 describing certain phases of the surgical procedure. For example, standardized description 208 may describe key surgical events (e.g., rare and/or unexpected events described further below) identified during information extraction stage 520. Standardized description 208 may include text describing the detected key surgical events, the identified anatomical structures identified, details associated with the identified anatomical structures, or other information. For example, portions of the generated text may be associated with one or more images to be included in the video surgical report. Language generation stage 620 may also take, as input, preoperative information 210.

Preoperative information 210 may include results of medical exams performed on the patient prior to the surgical procedure (e.g., blood tests). Preoperative information 210 may instead or additionally include preoperative imaging performed on the patient (e.g., X-Rays, CT scans, MRIs, etc.). One or more of the images captured prior to the surgical procedure being performed may be added to a later produced video surgical report (e.g., video surgical report 216 of FIG. 2). For example, one or more of trained machine learning models 412 of FIG. 4, trained machine learning models 512 of FIG. 5, or trained machine learning models 614 of FIG. 6 may be used to analyze images captured prior to the surgical procedure (e.g., preoperative imaging performed on patient of FIG. 1A). A similarity between the content depicted by the preoperative images and the content depicted by images from surgical video feed 202 may be determined. A determination may be made using trained machine learning models 412 of FIG. 4, trained machine learning models 512 of FIG. 5, and/or trained machine learning models 614 of FIG. 6 to determine whether the preoperative images and the images from surgical video feed 202 depict similar content. A similarity metric may be computed (e.g., based on an L2 distance, Manhattan distance, or other distance metric). The similarity metric may be used to determine whether the content depicted by two different images is “similar.” For example, a frame from surgical video feed 202 may be compared to a preoperative image, and a similarity metric for another image may be computed based on the comparison. If the computed similarity metric is less than a threshold similarity score, this may indicate that the frame from surgical video feed 202 depicts the same (or similar) content as the preoperative image. In this instance, the preoperative image may be added to video surgical report 216. Additionally or alternatively, the preoperative image and the frame from surgical video feed 202 may be presented to the user as an option for inclusion in video surgical report 216. Preoperative information 210 may instead or additionally include medical reports of previously performed surgical procedures of the patient.

Standardized description 208 may be provided as input to language translation stage 720. At language translation stage 720, standardized description 208 may be translated to obtain translated description 212. Language translation stage 720 may translate standardized description 208 from a first language to a second language. For example, standardized description 208 may include text in English, which may be translated to another language (e.g., English to French, English to Spanish, English to German, English to Japanese, etc.). Furthermore, language translation stage 720 may create multiple translated descriptions 212, each in a different language. Language translation stage 720 may be skipped or omitted if it is determined that standardized description 208 includes text in a language that does not need to be translated.

Standardized description 208 may be provided as input to audio generation stage 820. Optionally, translated description 212 may instead or additionally be provided as input to audio generation stage 820. At audio generation stage 820, audio data representing audio of the generated text (e.g., standardized description 208 and/or translated description 212) may be transformed into audio data 214. For example, a text-to-speech model may be used at audio generation stage 820 to generate audio data 214 representing the text of standardized description 208 and/or translated description 212. Audio generation stage 820 may employ an audio profile associated with a surgeon that performed the surgical procedure. For example, the audio profile may include data indicating a pitch, tone, accent, timbre, a loudness (volume of speech), a modulation associated with the user, and/or other characteristics of the surgeon's voice/speech.

Audio data 214 may be provided as input to video generation stage 920 to obtain a video surgical report 216. At video generation stage 920, the extracted images, video, description, audio, and/or other content may be aggregated into video surgical report 216. The video surgical report 216 may have a shorter duration than the surgical video feed 202. Thus, the video surgical report generation process 200 may include a temporal compression of the surgical video feed 202 with no, or minimal, loss of information. The extracted images, video, description, audio, and/or other content aggregated into video surgical report 216 can even provide that the surgical video feed 202 is temporally compressed, with increase of information content.

Video generation stage 920 may include steps for generating a virtual character programmed to output audio data 214. For example, the virtual character may be an avatar resembling the appearance of a surgeon (e.g., the surgeon that performed the surgical procedure). The virtual character may be generated using deep fake technology and/or other synthetic media. The virtual character may be programmed to speak audio data 214. For example, the facial expressions of the avatar may be used to recreate the facial movements of the surgeon. This may allow video surgical report 216 to present a comprehensive report detailing the surgical procedure using a virtual character of a user (e.g., surgeon, medical staff, etc.) that appears to dictate information about the surgical procedure.

Returning to FIG. 1B, information distillation subsystem 110 may be configured to access images captured during a surgical procedure, analyze those images using one or more machine learning models, and obtain distillation information 204 (shown in FIG. 2). For example, the images may be frames from surgical video feed 202. Distillation information 204 may be determined on a frame-by-frame basis. For example, for each frame of a video of a surgical procedure, distillation information 204 may be produced. Distillation information 204 may include an indication of a surgical phase depicted by a frame, a surgical activity or activities that occurred during the surgical phase, or other information, or combinations thereof.

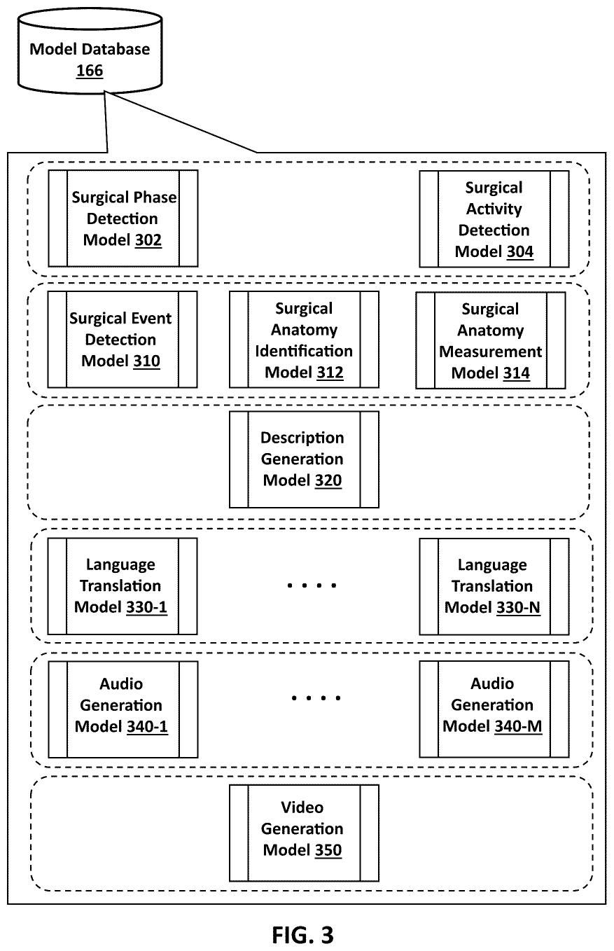

Information distillation subsystem 110 may be configured to train one or more machine learning models to produce distillation information (e.g., distillation information 204 in FIG. 2) based on one or more images. These images may include frames extracted from a surgical video feed (e.g., surgical video feed 202 in FIG. 2). For example, the surgical video feed may be captured using camera head 140 of endoscope 142, as seen in FIG. 1A. Different machine learning models may determine different types of distillation information. FIG. 3 includes examples of machine learning models that may be used by video surgical report generation process 200 to generate a surgical video report (e.g., video surgical report 216 in FIG. 2). For example, the models used to determine different types of distillation information may include a surgical phase detection model 302, a surgical activity detection model 304, and/or other models. Surgical phase detection model 302 may be trained to determine a surgical phase depicted by a given image. Surgical activity detection model 304 may be trained to determine a surgical activity occurring during the surgical phase.

With reference to FIG. 4, system 400 may include model training 410 and an information distillation stage 420. Model training 410 may include steps for training a machine learning model 402 retrieved from model database 166. Machine learning model 402 may be an instance of surgical phase detection model 302, surgical activity detection model 304, or another model related to information distillation stage 420 hat is to be trained. Alternatively, a single machine learning model 402 may be trained to perform the functions of each of surgical phase detection model 302 and surgical activity detection model 304.

Training data may be retrieved from training data database 164 (e.g., via information distillation subsystem 110) to train machine learning model 402. The training data may be selected from training data database 164 based on a type of model to be trained. For example, in the instance surgical phase detection model 302 is to be trained, the retrieved training data may include images 406a and 406b depicting various surgical phases. Images 406a and 406b may also include associated metadata. The metadata may indicate the particular surgical phases depicted by each of images 406a and 406b. Images 406a may include a first plurality of images representing a first surgical phase and images 406b may include a second plurality of images representing a second surgical phase. For example, the first plurality of images of images 406a may each depict an individual object or combination of objects, whereas the second plurality of images of images 406b may each depict a different object or combination of objects. Although only two sets of images are depicted within FIG. 4, persons of ordinary skill in the art will recognize that additional sets of images depicting similar or different surgical phases may also be included within the training data used to train machine learning model 402.

Objects depicted within images 406a and 406b may indicate the surgical phases. For example, certain individual objects and/or combinations of objects may be depicted by images captured during the first phase of the surgical procedure, while different objects and/or combinations of objects may depicted by images captured during the second phase of the surgical procedure.

As another example, in the instance surgical activity detection model 304 is to be trained, the retrieved training data may include images 406a and 406b depicting various surgical activities. These surgical activities may be performed during a detected surgical phase (e.g., a surgical phase detected by surgical phase detection model 302). Images 406a and 406b may also include associated metadata indicating the surgical activities. For example, images 406a may depict a first surgical activity performed during a first surgical phase and images 406b may depict a second surgical activity performed during the first surgical phase. Although only two sets of images are depicted within FIG. 4, persons of ordinary skill in the art will recognize that additional sets of images depicting similar or different surgical activities occurring during the same or different surgical phases may also be included within the training data and used to train machine learning model 402.

Objects depicted by images 406a and 406b may indicate the surgical activities being performed. Sequences of images 406a and 406b may describe movements of objects (e.g., medical staff) within medical environment 10 (shown in FIG. 1A). For example, an object may be tracked through a sequence of images to identify the object's motion, which may be used to determine the surgical activity that object is associated with.

Machine learning model 402 may be implemented using one or more machine learning architectures. For example, machine learning model 402 may be implemented as a convolutional neural network (CNN), a long short-term memory (LSTM) model, a temporal convolutional network (TCN), one or more vision transformers, another type of machine learning model, or a combination thereof. For example, machine learning model 402 may include an EndoNet, TeCNO, OperA, and/or Trans-SVNet model, each of which may correspond to known surgical phase detection models.

During model training 410, images 406a and 406b may be provided as input to machine learning model 402. Machine learning model 402 may predict a result based on the input images and values assigned to hyperparameters of machine learning model 402. For example, the predicted result may include a predicted surgical phase depicted by a given image from images 406a and 406b. As another example, the predicted result may include a predicted surgical activity occurring during the surgical phase based on the given image. The predicted result may be compared to the associated metadata for images 406a and 406b. The comparison may be used to compute a loss, which may then be minimized at 404. For example, a cross-entropy loss may be used at 404. The values of the hyperparameters of machine learning model 402 may be adjusted based on the loss. After the hyperparameters' values have been adjusted, additional images may be provided to machine learning model 402, new predictions may be made, new comparisons can be performed, and adjustments to some or all of the hyperparameters may be made. This process may repeat a predefined number of times and/or until a threshold accuracy level is achieved from machine learning model 402 (e.g., 75% or greater accuracy, 85% or greater accuracy, 95% or greater accuracy, etc.).

The trained version of machine learning model 402 (trained machine learning model 412) may be used for information distillation stage 420. During information distillation stage 420, image feed 408 may be retrieved from image database 162 (e.g., via information distillation subsystem 110 illustrated in FIG. 1B). Image feed 408 may include a sequence of images (e.g., forming a video). For example, a video captured by camera head 140 of endoscope 142 may be split into frames (e.g., via information distillation subsystem 110 illustrated in FIG. 1B), and the individual frames may be provided as input to trained machine learning model 412. Furthermore, while image feed 408 is depicted in FIG. 4 as being retrieved from image database 162, image feed 408 may alternatively be obtained directly from camera head 140 of endoscope 142.

Based on image feed 408, trained machine learning model 412 may output a surgical phase result 414, a surgical activity result 416, or other results. Surgical phase result 414 may indicate a predicted surgical phase depicted by image feed 408. Surgical phase result 414 may be a vector having n dimensions, where n equals a number of surgical phases that may occur during a surgical procedure. Surgical activity result 416 may indicate a predicted surgical activity depicted by image feed 408. Surgical activity result 416 may be based on surgical phase result 414. For example, a certain subset of surgical activities may typically occur during a first surgical phase, while another subset of surgical activities may typically occur during a second surgical phase. Surgical activity result 416 may be a vector having m dimensions, where m equals a number of surgical activities that may occur during the surgical procedure. Trained machine learning model 412 may output separate results, for example a separate surgical phase result 414 and surgical activity result 416. Alternatively, a single result may be output including each of surgical phase result 414 and surgical activity result 416. Therefore, for each input image from image feed 408, trained machine learning model 412, may determine the corresponding surgical phase result 414 and/or surgical activity result 416.

Trained machine learning model 412 may classify images from image feed 408 together based on surgical phase result 414 and/or surgical activity result 416. Trained machine learning model 412 may identify a subset of frames from image feed 408 (e.g., video of the surgical procedure) that depict objects associated with surgical phases, surgical activities, or other aspects of the surgical procedure. For example, for each image frame from image feed 408, a surgical phase may be determined by trained machine learning model 412, which may be indicated by surgical phase result 414. Image frames from image feed 408 that are determined to depict the same surgical phase (e.g., surgical phase result 414 may be the same or similar for multiple images from image feed 408) may be classified (e.g., grouped) together. As an example, image feed 408 may include a first image and a second image captured during a same phase of the surgical procedure. Trained machine learning model 412 may compute a first classification score for the first image and a second classification score for the second image. The first classification score may indicate how likely the first image depicts a particular surgical phase, and the second classification score may indicate how likely the second image depicts the same surgical phase. The first image and/or the second image may be added to a set of images which may be used for a video surgical report produced by video surgical report generation process 200 of FIG. 2.

Returning to FIG. 1B, information extraction subsystem 112 may be configured to extract information (e.g., extraction information 206) from surgical video feed 202 (e.g., illustrated in FIG. 2) based on distillation information 204. As mentioned above, distillation information 204 may be determined on a frame-by-frame basis. Therefore, information extraction subsystem 112 may receive distillation information 204 for each frame of surgical video feed 202.

Information extraction subsystem 112 may be configured to train one or more machine learning models to produce extraction information based on one or more images and distillation information 204 (shown in FIG. 2) associated with those images. These images may include frames extracted from surgical video feed 202. Different machine learning models may identify and extract different types of extraction information. As an example, with reference to FIG. 3, the extraction models may include a surgical event detection model 310, a surgical anatomy identification model 312, a surgical anatomy measurement model 314, or other models. Surgical event detection model 310 may be trained to detect key surgical events occurring during the detected surgical phase. For example, the key surgical events may include rare and/or unexpected events (e.g., accidental blood being detected, bile leakage, etc.). The key surgical events may also include detection of vessel ruptures. Key surgical events may also be detected based on data received from other aspects of medical environment 10 (shown in FIG. 1A). For example, electrocardiogram (ECG) data may be received from an ECG machine indicating that a myocardial infarction or other cardiac event occurred during the surgical procedure. As another example, brainwave activity (e.g., electroencephalogram (EEG)) data may be obtained from an EEG machine, which may indicate stroke, hemorrhage, or other neurological events occurred during the procedure. Surgical anatomy identification model 312 may be trained to identify a type of anatomical structure within an image. Surgical anatomy measurement model 314 may be trained to estimate parameters associated with detected anatomical structures and/or key surgical events. For example, surgical anatomy measurement model 314 may be used to estimate an anatomical structure's length, width, height, and/or volume, or other aspects of an anatomical structure identified within the image. As another example, surgical anatomy measurement model 314 may be used to estimate an amount of blood loss or an amount of other anatomical materials that may accumulate during the surgical procedure.

As an example, with reference to FIG. 5, system 500 may include a model training 510 and an information extraction stage 520. Model training 510 may include steps for training a machine learning model 502 retrieved from model database 166 (shown in FIG. 1B). Machine learning model 502 may be an instance of surgical event detection model 310, surgical anatomy identification model 312, surgical anatomy measurement model 314, or another model that is to be trained. Alternatively, a single machine learning model 502 may be trained to perform the functions of each of surgical event detection model 310, surgical anatomy identification model 312, and surgical anatomy measurement model 314.

Information extraction subsystem 112 (shown in FIG. 1B) may retrieve training data from training data database 164 to train machine learning model 502. The training data may be selected from training data database 164 based on a type of model to be trained. If, in the instance surgical event detection model 310 is to be trained, the retrieved training data may include images 506a and 506b depicting examples of key surgical events. For example, images 506a may include a first plurality of images each depicting a first key surgical event and images 506b may include a second plurality of images depicting a second key surgical event. Images 506a and 506b may also include associated metadata indicating the key surgical event depicted by those images.

In the instance surgical anatomy identification model 312 is to be trained, the retrieved training data may include images 506a and 506b depicting examples of various anatomical structures. For example, the first plurality of images (e.g., images 506a) may depict an individual anatomical structure and/or combinations of anatomical structures, and the second plurality of images (e.g., images 506b) may depict a different anatomical structure and/or combinations of anatomical structures. Images 506a and 506b may also include associated metadata indicating the particular anatomical structure depicted by each of images 506a and 506b, as well as any surgical events detected. For example, images 506a may depict an example of a first anatomical structure and images 506b may depict an example of a second anatomical structure.

In the instance surgical anatomy measurement model 314 is to be trained, the retrieved training data may include images 506a and 506b depicting examples of various anatomical structures, scales for measuring those anatomical structures, measurements of those anatomical structures (e.g., length, width, circumference, volume, etc.), or other information. Images 506a and 506b may also include associated metadata indicating the particular anatomical structure depicted by those images, as well as measurements or other information measured with respect to the anatomical structures. Although only two sets of images are depicted within FIG. 5, persons of ordinary skill in the art will recognize that additional sets of images may also be included within the training data used to train machine learning model 502 (e.g., to train one or more of surgical event detection model 310, surgical anatomy identification model 312, and/or surgical anatomy measurement model 314).

The training data may also include distillation information 508a and 508b, corresponding to images 506a and 506b, respectively. Distillation information 508a and 508b may include an indication of a surgical phase and/or surgical activity. For example, distillation information 508a and 508b may each include surgical phase result 414 and surgical activity result 416 (each of which are shown in FIG. 4) depicted by images 506a and 506b. Each image in images 506a and 506b may be associated with a respective surgical phase result and/or surgical activity in distillation information 508a and/or distillation information 508b.

Machine learning model 502 may be implemented using one or more machine learning architectures. For example, machine learning model 502 may be implemented as a convolutional neural network (CNN), a long short-term memory (LSTM) model, a temporal convolutional network (TCN), one or more vision transformers, a generative adversarial network (GAN), regressors, another type of machine learning model, and/or a combination thereof. For example, machine learning model 502 may include a You-Only-Look-Once (YOLO), Unet, Deeplab, LRASPP (based on MobileNet v3) model, each of which may correspond to known machine learning models for information extraction.

During model training 510, images 506a and 506b may be provided as input to machine learning model 502. Machine learning model 502 may predict a result based on images 506a and 506b, distillation information 508a and 508b, and values assigned to hyperparameters of machine learning model 502. The predicted result may include predicted extraction information (e.g., extraction information 206 shown in FIG. 2). For example, the predicted extraction information may include a prediction of a surgical event, a prediction of one or more anatomical structures, and/or a prediction of one or more measurements of the anatomical structures, each of which may be depicted by images 506a and 506b. The predicted extraction information may be compared to distillation information 508a and 508b, which may include predetermined information related to one or more of the aforementioned predictions and associated with each of image 506a and 506b. The comparison may be used to compute a loss, which may be minimized at 504. For example, a cross-entropy loss may be used at 504. The values of the hyper-parameters of machine learning model 502 may be adjusted, new images may be provided as input to machine learning model 502, new predictions may be made, new comparisons may be performed, and/or new adjustments may be made to the values of the hyperparameters of machine learning model 502. This process may repeat a predefined number of times and/or until a threshold accuracy level is achieved from machine learning model 502 (e.g., 75% or greater accuracy, 85% or greater accuracy, 95% or greater accuracy, etc.).

The trained version of machine learning model 502 (trained machine learning model 512) may be used at information extraction stage 520. During information extraction stage 520, image feed 514 may be retrieved (e.g., by information extraction subsystem 112 illustrated in FIG. 1B). Image feed 514 may be the same or similar to image feed 408, and the previous description may apply. For example, image feed 514 may be retrieved from image database 162 or directly from medical device 120 (e.g., an endoscope).

As mentioned above, machine learning model 502 may be trained to determine whether a given image depicts a key surgical event, whether the given image depicts one or more anatomical structures, whether anatomical structure measurement information can be derived, other information, and/or combinations thereof. The trained version of machine learning model 502 may correspond to trained machine learning model 512. At information extraction stage 520, information extraction subsystem 112 (shown in FIG. 1B) may be configured to execute trained machine learning model 512. Image feed 514 and distillation information 524 (obtained from the one or more machine learning models executed during information distillation stage 420 of FIG. 4) may be provided as input to trained machine learning model 512. Trained machine learning model 512 may output a key surgical event result 516, an anatomical structure result 518, an anatomical structure measurement result 522, and/or other results. Trained machine learning model 512 may output each of the aforementioned results separately as a set of one of more outputs (e.g., each output comprising one or more of the aforementioned results), and/or as a single output (e.g., comprising each of the aforementioned results). Trained machine learning model 512 may further be configured to extract one or more images 530 associated with some or all of the key surgical events, some or all of the anatomical structures, and/or some or all of the measurements for the anatomical structures. For example, trained machine learning model 512 may collect and temporally organize images from image feed 514 that are determined to depict a same (or similar) key surgical event (or a same/similar anatomical structure). Images 530 may correspond to a video and/or a snippet of a video (e.g., a snippet of a video of a surgical procedure).

Key surgical event result 516 may indicate one or more key surgical events depicted by image feed 514. The key surgical event may correspond to one of a plurality of key surgical events associated with the surgical procedure. Key surgical event result 516 may be an n-dimensional vector, where n indicates a number of key surgical events that may occur during the surgical procedure. Each element of the vector may include a score indicating a likelihood that trained machine learning model 512 identified one of the key surgical events from a predetermined set of known surgical events.

Anatomical structure result 518 may indicate one or more anatomical structures depicted by image feed 514. Anatomical structure result 518 may be based on key surgical event result 516. Based on the key surgical events detected, different subsets of anatomical structures may be expected to be visible within image feed 514. For example, during one key surgical event, a first subset of anatomical structures may be expected to be depicted by at least a portion of image feed 514, whereas during another (different) key surgical event, a second subset of anatomical structures may be expected to be depicted by at least another portion of image feed 514. Anatomical structure result 518 may be an m-dimensional vector, where m indicates a number of anatomical structures that may be detected during the surgical procedure from a known set of anatomical structures. Each element of the vector may include a score indicating a likelihood that trained machine learning model 512 identified one of the known anatomical structures.

Anatomical structure measurement result 522 may indicate an estimated size, shape, and/or other measurements associated with the anatomical structures depicted by image feed 514. Anatomical structure measurement result 522 may be based on anatomical structure result 518. Anatomical structure measurement result 522 may be a number, for example, a length, width, height, volume, etc., associated with the anatomical structure. Different units of measurement may be used to express anatomical structure measurement result 522. For example, the measurements may be expressed in centimeters, millimeters, inches, feet, etc.

Returning to FIG. 2, video surgical report generation process 200 may include a language generation stage 620. At language generation stage 620, text may be generated to describe one or more of the images associated with some or all of the key surgical events, some or all of the anatomical structures, and/or some or all of the measurements for the anatomical structures (determined as described above). The generated text may describe aspects of the key surgical events and may use extraction information 206 and/or preoperative information 210 to assist in the language formulation. In addition, text may be generated based on information regarding anatomical structures detected during key surgical events. For example, text may be generated describing an anatomical structure and/or describing measurements of the anatomical structure based on anatomical structure results 518 and/or anatomical structure measurement result 522 (shown in FIG. 5). Language generation stage 620 may output a standardized description 208 (shown in FIG. 2) including the generated text. Standardized description 208 may also include metadata indicating the particular images (e.g., images 530 illustrated in FIG. 5) associated with the text.

As mentioned above with respect to FIG. 1B, language generation subsystem 114 may be configured to train and execute one or more machine learning models for generating standardized description 208. Different machine learning models may be used to generate different types of descriptions. As an example, with reference to FIG. 3, the language generation models may include one or more description generation models 320. Description generation model 320 may be trained to generate text describing extraction information 206 (shown in FIG. 2). For example, the text may describe images, key surgical events, anatomical structures, and/or anatomical structure measurements identified at information extraction stage 520.

With reference to FIG. 6, system 600 may include a model training 610 and a language generation stage 620. Model training 610 may include steps for training a machine learning model 602 retrieved from model database 166 (shown in FIG. 1B). Machine learning model 602 may be an instance of description generation model 320, or another model that may be trained to generate a standardized description.

Training data may be received from training data database 164 (e.g., via language generation subsystem 114) to train machine learning model 602. The training data may be selected from training data database 164 based on a type of model to be trained. For example, for description generation model 320, the retrieved training data may include relevant information 606 and/or pre-generated text 608. Pre-generated text 608 may represent text generated based on relevant information 606. For example, relevant information 606 may include critical information describing a previously performed surgical procedure. Relevant information 606 may include information related to some or all of the key surgical events, some or all of the anatomical structures, and/or some or all of the measurements for the anatomical structures (determined as described above). Pre-generated text 608 may include descriptions manually generated by a surgeon or other medical professional associated with the medical procedure and/or descriptions generated using one or more machine learning models (e.g., prior instances of machine learning model 602, different machine learning models, etc.).

Machine learning model 602 may be implemented using one or more machine learning architectures. For example, machine learning model 602 may be implemented as a convolutional neural network (CNN), a generative adversarial network (GAN), or another type of machine learning model, or a combination thereof. An example machine learning model that may be used for machine learning model 602 includes the framework described by LSTM vs. GRU vs. Bidirectional RNN for script generation, to Mangal et al., 2018.

Model training 610 may provide machine learning model 602 with relevant information 606 and pre-generated text 608. Machine learning model 602 may generate a predicted description based on relevant information 606 and values assigned to hyperparameters of machine learning model 602. As an example, the predicted description may include text based on key surgical events identified, anatomical structures detected during those key surgical events, measurements of the anatomical structures, and/or other information. The predicted description may include one or more sentences written in a predetermined language (e.g., English, French, Spanish, German, Japanese, etc.). The predicted description may be compared to pre-generated text 608 to evaluate the performance of machine learning model 602 in generating contextually relevant text based on relevant information 606. The comparison may be used to compute a loss, which may then be minimized at 604. For example, a cross-entropy loss may be used at 604. The hyper-parameter values may be adjusted, and images may again be provided to machine learning model 602, whereby new predictions may be made and new comparisons may be performed, and additional adjustments may be made to the hyperparameters of machine learning model 602. This process may repeat a predefined number of times and/or until a threshold accuracy level is achieved from machine learning model 602 (e.g., 75% or greater accuracy, 85% or greater accuracy, 95% or greater accuracy, and the like).

The trained version of machine learning model 602 (analogous to trained machine learning model 614) may be used at language generation stage 620. During language generation stage 620, one or more images 530 may be retrieved (e.g., via language generation subsystem 114 shown in FIG. 1B), as shown in FIG. 6. As mentioned with reference to FIG. 5, images 530 may correspond to images associated identified key surgical events (e.g., key surgical event results 516). Images 530 may include video snippets from the overall video of the surgical procedure and/or subsets thereof, sets of images, and/or individual images. Furthermore, language generation subsystem 114 may be configured to provide relevant information 612 and/or images 530 to trained machine learning model 614. Relevant information 612 may include extraction information 206 and/or preoperative information 210 (each of which are shown in FIG. 2). For example, relevant information 612 may include indications of key surgical events depicted by images 530, anatomical structures identified within images 530, measurements of the identified anatomical structures, or other information. Preoperative information 210 may include previous medical exam results associated with the patient, medical images captured prior to the current surgical procedure, and/or other medical notes/information associated with the patient, the surgeon, and/or the surgical procedure.

Based on the input comprising relevant information 612 and/or images 530, trained machine learning model 614 may output generated text 616. For example, standardized description 208 may include generated text 616. Generated text 616 may also include images 618. Images 618 may correspond to some or all of images 530. Trained machine learning model 614 may determine whether any images 530 are redundant or not needed to describe a particular aspect of the surgical procedure. For example, if two or more of images 530 depict the same content, then trained ML model 614 may remove all but one of those images from images 530 to obtain images 618. The user-provided text data may be compared to pre-generated text data associated with relevant information 612. For example, relevant information 612 may include pre-generated text data comprising an indication of the content depicted by one or more images from surgical video feed 202 (shown in FIG. 2). In this example, generated text 616 may be generated based on a comparison of the user-provided text data and the pre-generated text data. The comparison may weigh the user-provided text data and the pre-generated text data differently. For example, the user-provided text data may be weighted as more relevant than the pre-generated text data. Therefore, trained machine learning model 614 may be trained to incorporate more of the user-provided text data into generated text 616 and less of the pre-generated text data based on the weights of each of the types of text data.

Returning to FIG. 2, video surgical report generation process 200 may include language translation stage 720, which may follow language generation stage 620. Language translation stage 720 may be employed to produce translated description 212 when a surgical report is to be generated in a language other than a pre-programmed default language of language generation stage 620. Alternatively or additionally, language translation stage 720 may be employed when additional surgical reports in other languages are to be produced. For example, multiple language models may be used to create translated descriptions 212. In an example, language generation stage 620 and language translation stage 720 may be combined. For example, surgical video feed 202, distillation information 204, extraction information 206, preoperative information 210, or other information may be used to generate standardized description 208 and/or one or more translated descriptions 212 at a single stage. As shown in FIG. 2, language translation stage 720 may be optional within video surgical report generation process 200, as indicated by the dashed lines.

As mentioned above with respect to FIG. 1B, language translation subsystem 116 may be configured to train and execute one or more machine learning models for translating a description produced in one language (e.g., standardized description 208 of FIG. 2) into one or more translated descriptions in one or more other languages (e.g., translated description 212). Different machine learning models may generate descriptions in different languages. As an example, with reference to FIG. 3, the language translation models may include a first language translation model 330-1 to an N-th language translation model 330-N (which collectively may be referred to as language translation models 330). For example, language translation models 330 may translate descriptions into include English, French, Spanish, Italian, Portuguese, German, Japanese, Mandarin, Cantonese, Korean, and/or other languages.

As an example, with reference to FIG. 7, system 700 may include a model training 710 and a language translation stage 720. Model training 710 may include steps for training a machine learning model 702 retrieved from model database 166 (shown in FIG. 1B). Machine learning model 702 may be an instance of one of language translation models 330 (e.g., one of first language translation model 330-1 to N-th language translation model 330-N) shown in FIG. 3 that is to be trained.

Training data from training data database 164 to train machine learning model 702 of FIG. 7. The training data may be selected from training data database 164 may be retrieved (e.g., via language translation subsystem 116 illustrated in FIG. 1B) based on a type of model to be trained. For example, in the instance the source language (e.g., the original language) is English and the target language is French, the training data may include data for English text and French text corresponding to the English text. Original language text 706 and target language text 708 may be provided as input to machine learning model 702 to train machine learning model 702 to translate text from the original language to the target language. In addition to original language text 706 and target language text 708, relevant information 606, which previously was described as an input to machine learning model 602 shown in FIG. 6, may be provided as input to machine learning model 702.

Machine learning model 702 may be implemented using one or more machine learning architectures. For example, machine learning model 702 may be implemented as a convolutional neural network (CNN), a recurrent neural network (RNN), a long short-term memory (LSTM) model, an LSTM plus Attention model, a transformer, generative adversarial network (GAN), another type of machine learning model, or a combination thereof. An example machine learning model that may be used for machine learning model 702 includes the framework described by “Deep contextualized word representations,” to Peters et al., 2018 and/or “BERT: Pre-training of Deep Bidirectional Transformers for Language Understanding,” to Devlin et al., 2018.

Model training 710 may provide machine learning model 702 with relevant information 606, original language text 706, and target language text 708, and/or other information. Machine learning model 702 may generate predicted text in the target language based on original language text 706, relevant information 606, and values assigned to hyperparameters of machine learning model 702. The predicted text may include a translated description (e.g., translated description 212 shown in FIG. 2). The translated description may include one or more sentences written in a target language. The predicted text may be compared to target language text 708 to determine evaluate the performance of machine learning model 702 in translating original language text 706. The comparison may be used to compute a loss, which may then be minimized at 704. For example, a cross-entropy loss may be used at 704. The values of the hyper-parameters may be adjusted based on the loss. Additional original language text 706 may be input to machine learning model 702 after the hyperparameters have been adjusted, and new predicted descriptions may be generated, new comparisons may be performed, and additional adjustments to the hyperparameters may be made. This process may repeat a predefined number of times and/or until a threshold accuracy level is achieved from machine learning model 702 (e.g., 75% or greater accuracy, 85% or greater accuracy, 95% or greater accuracy, etc.).

The trained version of machine learning model 702 (analogous to trained machine learning model 714) may be used at language translation stage 720. During language translation stage 720, a description 712 and/or images 530 may be retrieved (e.g., via language translation subsystem 116 illustrated in FIG. 1B). Description 712 may be provided to trained machine learning model 714 and trained machine learning model 714 may produce generated text 716 in the target language. Description 712 may be in a same (or similar) language as original language text 706. As mentioned above, images 530 may correspond to images associated with identified key surgical events (e.g., key surgical event results 516 of FIG. 5). Images 530 may include video snippets from the overall video of the surgical procedure or subsets thereof, sets of images, and/or individual images.

Generated text 716 may include images 718 in addition to text in the target language. Images 718 may include some or all of images 530. In an example, images 718 may include some or all of images 618 of FIG. 6. Metadata may also be stored with generated text 716 (e.g., analogous to translated description 212 shown in FIG. 2) indicating which words and/or phrases in the target language correspond to which words and/or phrases in the original language.

Returning to FIG. 2, video surgical report generation process 200 may include audio generation stage 820, which may follow language generation stage 620 and/or language translation stage 720 (e.g., in the instance language translation stage 720 is included in process 200). For example, translated description 212 may be used to generate audio data 214 representing an audio description for the video surgical report in the target language. For example, standardized description 208 and/or translated description 212 may be used to generate audio data 214 representing an audio description for the video surgical report in a desired language.

As mentioned above with respect to FIG. 1B, audio generation subsystem 124 may be configured to train and execute one or more machine learning models for generating audio representing standardized description 208 and/or translated description 212. Different machine learning models may be used to generate audio in different languages. As an example, with reference to FIG. 3, the audio generation models may include a first audio generation model 340-1 to an M-th audio generation model 340-M (which collectively may be referred to as audio generation models 340). Each of audio generation models 340 may generate audio in a particular language. For example, audio generation models 340 may include English, French, Spanish, Italian, Portuguese, German, Japanese, Mandarin, Cantonese, Korean, and/or other languages.

As an example, with reference to FIG. 8, system 800 may include a model training 810 and an audio generation stage 820. Model training 810 may include steps for training a machine learning model 802 retrieved from model database 166 (shown in FIG. 1B). Machine learning model 802 may be an instance of one of audio generation models 340 (shown in FIG. 3) that is to be trained. Training data may be retrieved from training data database 164 (e.g., via audio generation subsystem 124 illustrated in FIG. 1B) to train machine learning model 802. The training data may be selected from training data database 164 based on a type of model to be trained. For example, in the instance machine learning model 802 is configured to generate audio in English, the training data may include text descriptions in English and audio generated for the description in English.

Machine learning model 802 may be implemented using one or more machine learning architectures. For example, machine learning model 802 may be implemented as a convolutional neural network (CNN), generative adversarial network (GAN), other text-to-speech (TTS) models, and/or a combination thereof. An example machine learning model that may be used for machine learning model 802 includes the GANSynth framework.

At model training 810, machine learning model 802 may be provided with text description 806. Text description 806 may be similar to standardized description 208 output at language generation stage 620 or translated description 212 output at language translation stage 720, as shown in FIG. 2. Machine learning model 802 may generate predicted audio based on text description 806 and values assigned to hyperparameters of machine learning model 802. The predicted audio may include spoken phrases corresponding to some or all of text description 806. An audio profile of a surgeon that performed the surgical procedure for which the video surgical report is generated, a patient to whom the surgical procedure was performed, or another medical professional associated with the surgical procedure, may also be provided to machine learning model 802. The audio profile may include data representing sound characteristics to be used when generating the audio. For example, the audio profile may indicate a tone, accent, etc. associated with the surgeon so as to produce audio that mimics the surgeon speaking.

The predicted audio may be compared to target audio 808 to evaluate the performance of machine learning model 802 in generating audio for text description 806. The comparison may be used to compute a loss, which may then be minimized at 804. For example, a cross-entropy loss may be used at 804. Values of the hyperparameters of machine learning model 802 may be adjusted and additional text descriptions (e.g., text descriptions 806) may be provided as input to machine learning model 802. Predicted audio may be generated for the additional text descriptions and new comparisons may be performed, which may be used to determine adjustments to the values of the hyperparameters of machine learning model 802. This process may repeat a predefined number of times and/or until a threshold accuracy level is achieved from machine learning model 802 (e.g., 75% or greater accuracy, 85% or greater accuracy, 95% or greater accuracy, etc.).

The trained version of machine learning model 802 (analogous to trained machine learning model 812) may be used at audio generation stage 820. During audio generation stage 820, text description 814 may be retrieved (e.g., via audio generation subsystem 124) and provided to trained machine learning model 812 to produce generated audio 816. Generated audio 816 may be analogous to audio data 214 output from audio generation stage 820, as shown in FIG. 2. Generated audio 816 may be in a selected language. For example, a notification may be received by the user comprising a request for an indication of a target language for the video surgical report. Based on the indicated language request, audio generation subsystem 124 of FIG. 1B may select a corresponding machine learning model (e.g., one or more of audio generation model 340-1 to audio generation model 340-M) trained to generate audio in the target language for trained machine learning model 812.

Returning to FIG. 2, video surgical report generation process 200 may include video generation stage 920, which may follow audio generation stage 820. Video generation stage 920 may generate video surgical report 216 based on audio data 214, extraction information 206, preoperative information 210, surgical video feed 202, standardized description 208, and/or translated description 212. As mentioned above with respect to FIG. 1B, video generation subsystem 126 may be configured to train and execute one or more machine learning models for generating video surgical reports based on audio data 214, extraction information 206, standardized description 208, and/or translated description 212. As an example, with reference to FIG. 3, the machine learning models for generating videos (e.g., video surgical reports) may include video generation model 350.

With reference to FIG. 9, system 900 may include a model training 910 and a video generation stage 920. Model training 910 may include steps for training a machine learning model 902 retrieved from model database 166. Machine learning model 902 may be an instance of video generation model 350 (shown in FIG. 3) that is to be trained. Training data may be retrieved from training data database 164 (e.g., via video generation subsystem 126 shown in FIG. 1B) to train machine learning model 902. The training data may include one or more of text descriptions 906, audio data 908, pre-generated videos 912, images 530, and, optionally, virtual character data 914. Machine learning model 902 may generate predicted video surgical reports based on the training data and values assigned to hyperparameters of machine learning model 902. The predicted video may include some or all of the images and/or videos (e.g., analogous to images 530 of FIG. 5) associated with key surgical events, anatomical structures, or other surgical activities. The predicted video may include text descriptions 906 generated based on images and/or videos of key surgical events, as well as audio generated based on the text descriptions (e.g., audio data 908). Text description 906 may be analogous to standardized description 208 of FIG. 2 and/or translated description 212. Audio data 908 may be analogous to audio data 214 of FIG. 2.

As mentioned above, virtual character data 914 may be used to assist in generating the video surgical report. For example, virtual character data 914 may be used to generate a virtual character (e.g., an avatar) that will be rendered in the video surgical report. The video surgical report may include data programmed to cause the virtual character to execute life-like motions and appear as though the virtual character is speaking. For example, virtual character data 914 may include programming describing how facial expressions of the user (e.g., surgeon or another medical professional) change when different phenomes are uttered, so as to re-create real-life facial movements. For example, the data may cause facial features of the virtual character to move while audio data 908 is output. Virtual character data 914 may include surgeon profile information associated with the surgeon or another medical professional associated with the surgical procedure. Using the surgical profile information, the video surgical report may include an avatar of the surgeon that performed the surgery, and the surgeon avatar may appear to talk to the patient viewing the video surgical report. For example, the avatar may describe the key surgical events that transpired during the surgery, which may enable the video surgical report to communicate key elements of the patient's health more effectively. The video surgical report may be provided to the patient for subsequent review after the surgical procedure has been completed (e.g., while the patient is recovering).

The predicted video may be compared to one or more pre-generated videos 912 to evaluate the performance of machine learning model 902 in generating video surgical reports based on the input text description, audio, virtual character data, and/or images of key surgical events. The comparison may be used to compute a loss, which may then be minimized at 904. For example, a cross-entropy loss may be used at 904. The hyper-parameter values may be adjusted and additional text descriptions 906, audio data 908, virtual character data 914, and/or images 530 may be provided as input to machine learning model 902. Predicted video surgical reports may be generated for the additional text descriptions, audio data, virtual character data, and/or images, and new comparisons may be performed. The values of the hyperparameters may be adjusted further based on the comparisons. This process may repeat a predefined number of times and/or until a threshold accuracy level is achieved from machine learning model 902 (e.g., 75% or greater accuracy, 85% or greater accuracy, 95% or greater accuracy, etc.).

In an example, machine learning model 902 may be implemented with a multi-modal, multi-task, multi-embodiment agent using a single neural model across different tasks. For example, machine learning model 902 may include the model described in “A Generalist Agent,” to Reed et al., November 2022, the disclosure of which is incorporated by reference in its entirety. For example, machine learning model 902 may be trained on data from different tasks and different modalities. The data may be serialized into a flat sequence of tokens, batched, and processed. In an example, a transformer neural network may be used. Masking may also be used such that the loss function may be applied only to its target outputs.

The trained version of machine learning model 902 (analogous to trained machine learning model 924) may be used at video generation stage 920. During video generation stage 920, text description 916, audio data 918 associated with text descriptions 916, and some or all of images 530 associated with text description 916 may be retrieved (e.g., via video generation subsystem 126). Additionally, video generation subsystem 126 may retrieve virtual character data 922 associated with a surgeon who performed and/or is associated with the surgical procedure for which the video surgical report is made. Video generation subsystem 126 may provide text description 916, audio data 918 associated with text descriptions 916, some or all of images 530 associated with text description 916, and virtual character data 922 to trained machine learning model 924, which may output video surgical report 926. Video surgical report 926 may be analogous to video surgical report 216 output from video generation stage 920 of video surgical report generation process 200 shown in FIG. 2. Video generation subsystem 126 may be configured to provide video surgical report 926 to client device 130 associated with a patient, a surgeon, and/or other medical professionals. Furthermore, video surgical report 926 may be stored in surgical report database 172. Video surgical report 926 may include metadata indicating a surgeon that performed the surgical procedure (e.g., using a surgeon name, employee number, and/or another identifier), a patient to whom the surgical procedure was performed (e.g., based on a patient identifier), and/or other identifying information.

In some examples, machine learning model 924 may produce video surgical report 926 based on images 530 (which may include videos), pre-generated videos 912, and/or virtual character data 914. In this example, generated audio 816 produced at audio generation stage 820 (shown in FIG. 8) may be combined with video surgical report 926. Generated audio 816 may be played, or otherwise dubbed, over video surgical report 926 output by trained machine learning model 924. In this example, generated audio 816 may be combined with video surgical report 926 output by trained machine learning model 924.

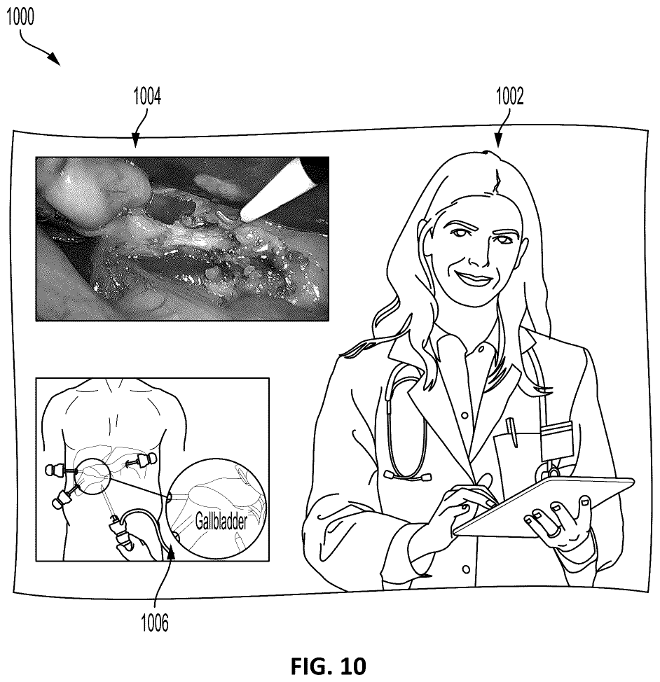

FIG. 10 illustrates an example video surgical report 1000, according to some aspects. Video surgical report 1000 may include a virtual character 1002, one or more images 1004, graphics 1006, and/or other content. Virtual character 1002 may be programmed such that the facial features of virtual character 1002 adjust to synchronize with audio (e.g., audio data 214). These adjustments may allow virtual character 1002 to appear as though it is speaking. Virtual character 1002 may be programmed such that it has a same or similar appearance as the surgeon or other medical professional associated with the surgical procedure that was performed. For example, virtual character 1002 may comprise similar facial characteristics, body type, hair style, hair color, similar scrubs and/or logo representing the medical facility, etc.

Images 1004 may include some or all of images 530. For example, images 1004 may include a subset of images 530 that depict key surgical events, identified anatomical structures, and/or other aspects of the surgical procedure. Images 1004 may instead or additionally include videos and/or video snippets of key surgical events. Images 1004 may be selected for presentation within video surgical report 1000 such that they synchronize with the audio being output during surgical report 1000. In addition to or instead of images 1004, graphics 1006 may be included within video surgical report 1000. Graphics 1006 may include pre-generated images, images extracted from a surgical video feed, and/or other content that can be presented to help explain or detail aspects of the surgical procedure. For example, graphics 1006 may include an animation associated with a key surgical event depicted by images 1004. Video surgical report 1000 may include two or more instances of images 1004, and each of these images may include graphics. For example, a pre-generated image of a particular anatomical structure may be presented within video surgical report 1000 in addition to an image extracted the surgical video feed determined to depict the same anatomical structure.

In some examples, video surgical report 1000 may be a component of a surgical report application (e.g., a mobile application, web application, etc.) that facilitates interactions with a patient. The surgical report application may communicate with a chatbot or other virtual communications system that enables a user, such as the patient, to input comments and/or questions for a medical professional and receive responses to those comments and/or questions. As an example, a user (e.g., a patient reviewing video surgical report 1000 on client device 130) may input a text query in a field of a user interface displaying video surgical report 1000. As another example, the user may speak an utterance that is detected by a speech recognition engine. The surgical report application may be configured to receive the input(s) and provide a response to the input(s). The response may be based on data associated with the surgical procedure, the patient's metadata, or may be based on other factors. For example, multimodal video question and answering techniques can be used by integrating a patient's electronic medical record information with data representing video surgical report 1000. Additionally, a knowledge-based video question and answering approach may use explicit data source-specific guidelines for the surgical procedure type to formulate the response to the user's input.

The surgical report application may be configured to generate audio and/or video of the response, which may be provided to the user within a user interface (e.g., a user interface presented on a display screen of a desktop computer, mobile phone, tablet, etc.) presenting video surgical report 1000. The surgical report application may be configured to program virtual character 1002 such that it appears to speak the generated response to the user input. For example, the surgical report application may generate a response to a question based on the above-mentioned question and answering techniques/approaches. The generated response may be text-based, and thus speech recognition processing techniques (e.g., such as those described above with respect to description generation model 320) may be used to generate audio data representing the response text (e.g., audio data 214 shown in FIG. 2). The surgical report application may instead or additionally generate video data representing the audio data, where the video data may include programming instructions for effectuating facial movements of virtual character 1002 synchronized with the audio data. This may allow the patient to ask “their surgeon” (e.g., when virtual character 1002 is programmed to be an avatar of the surgeon) questions about the surgical procedure and receive human-like responses from virtual character 1002.

An option may be provided for a user to override one or more outputs of the machine learning models used in video surgical report generation process 200 of FIG. 2. A surgical report application may allow a user to provide modifications to the outputs produced at one or more stages of video surgical report generation process 200. For example, the user may edit auto-generated text, add their own findings and/or observations, add/remove images and/or video fragments from the video surgical report, and/or perform other functions using the surgical report application. The user may then re-generate the video surgical report based on the updates.

Additional images and/or videos may also be captured after the surgical procedure, which may be used to generate an updated version of video surgical report 216. For example, post-surgical medical imaging may be performed on the patient (e.g., patient 12 of FIG. 1A). The additional images and/or videos may be added to video surgical report 216. The user may be able to add one or more of the additional images/videos to video surgical report 216 using the surgical report application.

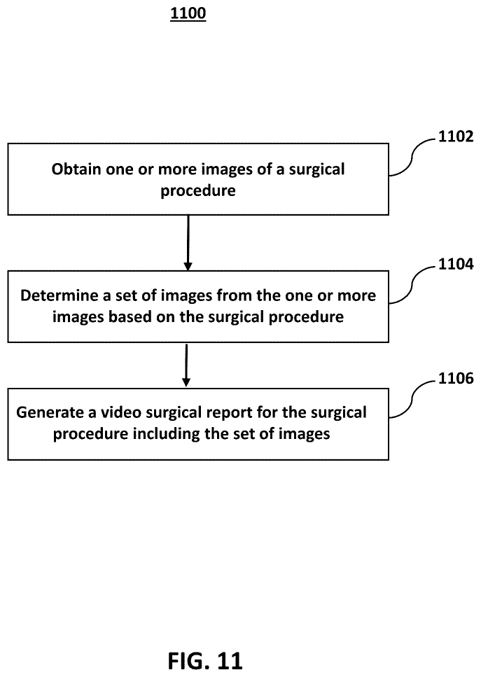

FIG. 11 illustrates an example flowchart of a method 1100 for generating a video surgical report, according to some aspects. At step 1102, one or more images of a surgical procedure may be obtained. The images may be captured via a medical device during a surgical procedure (e.g., via an endoscope). The images may be a portion of a surgical video feed captured by the medical device. The images may be a portion of a surgical video feed obtained from a memory, the images having been captured via or by the medical device. The images may have been captured prior to start of the method for generating the video surgical report. The images may include frames extracted from the surgical video feed. The surgical video feed may be split into frames (e.g., images) of the surgical procedure and stored in image database 162 (shown in FIG. 1B). To perform video surgical report generation process 200 shown in FIG. 2, the frames of surgical video feed 202 may be accessed and analyzed using one or more machine learning models, as described above. Some or all of the operations described at step 1102 may be performed by a subsystem that is the same or similar to information distillation subsystem 110.

At step 1104, a set of images from the obtained images may be determined based on the surgical procedure. The set of images may be selected based on one or more machine learning models. For example, the obtained images may be provided to one or more machine learning models during an information distillation stage 420. At information distillation stage 420, machine learning models, such as surgical phase detection model 302 and/or surgical activity detection model 304 shown in FIG. 3, may be used to analyze the obtained images (e.g., frames of the video surgical report). As seen in FIG. 2, information distillation stage 420 may output distillation information 204 (e.g., surgical phase result 414, surgical activity result 416 in FIG. 4), which may be passed to information extraction stage 520. At information extraction stage 520, the set of images (e.g., frames) may be extracted from those forming the surgical video feed based on distillation information 204 and using one or more machine learning models. For example, the frames forming surgical video feed 202, distillation information 204 (e.g., surgical phase result 414, surgical activity result 416), and/or other information may be provided to one or more machine learning models trained to obtain extraction information 206 and/or images, video snippets, etc. from surgical video feed 202. The one or more machine learning models may include surgical event detection model 310, surgical anatomy identification model 312, and/or surgical anatomy measurement model 314. The machine learning models may output, on a frame-by-frame basis, key surgical event result 516, anatomical structure result 518, anatomical structure measurement result 522, and/or other results. Furthermore, one or more images (e.g., video snippets) may be extracted based on the detected key surgical events, detected anatomical structures, and/or anatomical structure measurement information. This set of images may be used to generate a video surgical report. For example, as seen in FIG. 10, images 1004 may include one or more of images 530 (shown in FIG. 5). Some or all of the operations described at step 1104 may be performed by a subsystem that is the same or similar to information extraction subsystem 112.

At step 1106, a video surgical report may be generated for the surgical procedure, the video surgical report including the set of images. The video surgical report may be generated using one or more machine learning models. For example, machine learning models may be used to generate text describing the key surgical events, anatomical structures, etc., associated with a surgical activity occurring during a particular surgical phase. The machine learning models may instead or additionally be used to generate audio of the text, which may be produced in one or more languages. The machine learning models may also be used to generate the video surgical report based on the generated audio, the generated text, and/or other information distilled and extracted from the surgical video feed and/or preoperative information.

As an example, at language generation stage 620 of FIG. 6, relevant information 606 (e.g., images 530, key surgical event results 516, anatomical structure results 518, anatomical structure measurement results 522 shown in FIG. 5), and/or other data may be provided as input to one or more machine learning models (e.g., trained machine learning model 614). The machine learning models employed at language generation stage 620 may include description generation model 320. Description generation model 320 may be trained to receive relevant information 612 associated with images 530 and output generated text 616, which may include images 618. For example, images 530 may also be provided to description generation model 320 to form generated text 616. Images 618 of generated text 616 may include some or all of images 530. Generated text 616 may include words, phrases, sentences, etc. in a first language. For example, the first language may be English, German, French, Japanese, Spanish, etc. Description generation model 320 may be trained to generate text in a particular language. In the instance generated text 616 is to be generated in a different language, it may be translated at language translation stage 720 (shown in FIGS. 2 and 7). Some or all of the operations described at step 1106 may be performed by a subsystem that is the same or similar to language generation subsystem 114, language translation subsystem 116, or both.

As another example, at audio generation stage 820, standardized description 208 and/or translated description 212 may be provided as input to one or more machine learning models (e.g., trained machine learning model 812 of FIG. 8). The machine learning models employed at audio generation stage 820 may include audio generations models 340-1 to 340-M. Each audio generation model may be trained to generate audio in a particular language. Therefore, a particular machine learning model may be selected for audio generation based on the language of the input description (e.g., standardized description 208, translated description 212, etc.). Trained machine learning model 812 may generate audio data 214 based on the input description, as well as an audio profile of a surgeon that performed the surgical procedure. For example, the audio profile may include the pitch, accent, tone, etc., of the surgeon such that the generated audio appears as though the surgeon spoke it. Some or all of the operations described at step 1106 may be performed by a subsystem that is the same or similar to audio generation subsystem 124.

As another example, at video generation stage 920, standardized description 208, translated description 212, extraction information 206, distillation information 204, and/or other information, may be provided as input to one or more machine learning models (e.g., trained machine learning model 924 shown in FIG. 9). The machine learning models employed at video generation stage 920 may include one or more video generation models 350. The video surgical report may be generated such that it includes images captured from the surgical procedures, text describing aspects of the images, video snippets from the surgical video feed relating to the described aspects, the generated audio, virtual characters used to convey the audio to the patient, and/or other information. For example, trained machine learning model 924 may receive text description 916, audio data 918, and images 530 to create video surgical report 926. Trained machine learning model 924 may also receive virtual character data 922. Virtual character data 922 may include computer programming instructions for configuring a virtual character to be presented within video surgical report 926. For example, virtual character data 922 may include information describing attributes of a surgeon who performed the surgical procedure (e.g., appearance, dress, facial features, etc.). For example, characteristics of how the surgeon's face moves and changes when speaking different phonemes may be included in virtual character data 922 to allow the virtual character to appear life-like and interactive.

Video surgical report 926 may be presented within a user interface of a surgical report application. The surgical report application may interface with synthetic speech, audio, and video programs to enable a user (e.g., a patient) to input questions/comments and/or receive feedback to those input questions/comments. Video surgical report 926 may be presented to patient 12 using client device 130 (e.g., shown in FIG. 1B). For example, client device 130 may include a desktop computer, mobile phone, tablet, etc., which may include a display screen. A mobile application and/or web application may be rendered on the display screen so that patient 12 may review video surgical report 926.

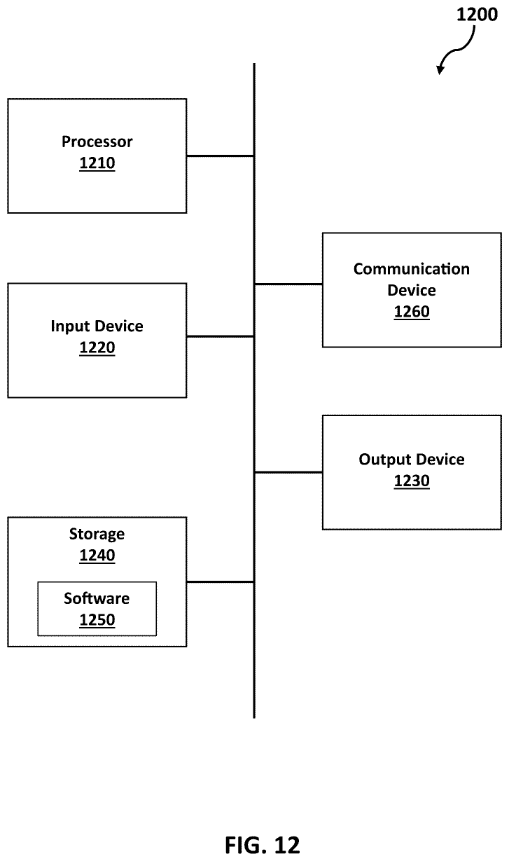

FIG. 12 illustrates an example computing system 1200, according to some aspects. Computing system 1200 may be used for performing any of the methods described herein, including method 1100 of FIG. 11, and can be used for any of the systems described herein, including computing system 102 (and the subsystems included therein), medical device 120, client device 130, or other systems/devices described herein. Computing system 1200 can be a computer coupled to a network, which can be, for example, an operating room network or a hospital network. Computing system 1200 can be a client computer or a server. As shown in FIG. 12, computing system 1200 can be any suitable type of controller (including a microcontroller) or processor (including a microprocessor) based system, such as an embedded control system, personal computer, workstation, server, or handheld computing device (portable electronic device) such as a phone or tablet. The system can include, for example, one or more of processor 1210, input device 1220, output device 1230, storage 1240, or communication device 1260.

Input device 1220 can be any suitable device that provides input, such as a touch screen, keyboard or keypad, mouse, gesture recognition component of a virtual/augmented reality system, or voice-recognition device. Output device 1230 can be or include any suitable device that provides output, such as a touch screen, haptics device, virtual/augmented reality display, or speaker.

Storage 1240 can be any suitable device that provides storage, such as an electrical, magnetic, or optical memory including a RAM, cache, hard drive, removable storage disk, or other non-transitory computer readable medium. Communication device 1260 can include any suitable device capable of transmitting and receiving signals over a network, such as a network interface chip or device. The components of the computer can be coupled in any suitable manner, such as via a physical bus or wirelessly.

Software 1250, which can be stored in storage 1240 and executed by processor 1210, can include, for example, the programming that embodies the functionality of the present disclosure (e.g., as embodied in the devices as described above). For example, software 1250 can include one or more programs for performing one or more of the steps of the methods disclosed herein.

Software 1250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1240, that can contain or store programming for use by or in connection with an instruction execution system, apparatus, or device.

Software 1250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic, or infrared wired or wireless propagation medium.

Computing system 1200 may be coupled to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 1200 can implement any operating system suitable for operating on the network. Software 1250 can be written in any suitable programming language, such as C, C++, C#, Java, or Python. In various examples, application software embodying the functionality of the present disclosure can be deployed in different configurations, such as in a client/server arrangement or through a Web browser as a Web-based application or Web service, for example.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,” “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively.

The foregoing description, for the purpose of explanation, has been described with reference to specific aspects. However, the illustrative discussions above are not intended to be exhaustive or to limit the invention to the precise forms disclosed. Many modifications and variations are possible in view of the above teachings. The aspects were chosen and described in order to best explain the principles of the techniques and their practical applications. Others skilled in the art are thereby enabled to best utilize the techniques and various aspects with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A computer-implemented method, comprising:
obtaining one or more images of a surgical procedure;
determining, using one or more machine learning models, a set of images from the one or more images based on the surgical procedure; and
generating a video surgical report for the surgical procedure comprising at least some of the set of images.

2. The method of claim 1, wherein generating the video surgical report comprises:
generating, using the one or more machine learning models, text describing the at least some of the set of images, wherein the video surgical report comprises at least some of the text corresponding to the at least some of the set of images.

3. The method of claim 1, wherein generating the video surgical report comprises:
generating a virtual character programmed to output audio associated with the at least some of the set of images, the video surgical report comprising the virtual character.

4. The method of claim 1, wherein determining the set of images comprises:
determining, using the one or more machine learning models, at least one of: a phase of the surgical procedure, a surgical activity being performed during the phase, or information related to the set of images; and selecting the set of images from the one or more images based on the at least one of the phase, the surgical activity, or the information related to the set of images.

5. The method of claim 4, further comprising:
training the one or more machine learning models to analyze content depicted by the set of images to determine the at least one of the phase, the surgical activity, or the information related to the set of images.

6. The method of claim 1, wherein the one or more images include a first image and a second image captured during a same phase of the surgical procedure, determining the set of images comprises:
computing, using the one or more machine learning models, a first classification score and a second classification score respectively associated with the first image and the second image; and
adding at least one of the first image or the second image to the set of images based on the first classification score and the second classification score.

7. The method of claim 1, further comprising:
identifying at least one image from the one or more images based on preoperative information related to the surgical procedure, wherein the set of images comprises the at least one image.

8. The method of claim 1, wherein obtaining the one or more images comprises:
identifying, using the one or more machine learning models, a subset of frames from a video of the surgical procedure that depict one or more objects associated with the surgical procedure, wherein the one or more images comprise the subset of frames.

9. The method of claim 1, further comprising:
detecting, using the one or more machine learning models, one or more objects associated with the surgical procedure within the one or more images, wherein the at least some of the set of images are selected based on the one or more objects.

10. The method of claim 1, further comprising:
receiving preoperative information related to at least one of the surgical procedure or a patient associated with the surgical procedure; and
generating content to be included in the video surgical report based on the received preoperative information.

11. The method of claim 1, further comprising:
generating, using the one or more machine learning models, text associated with the set of images; and
associating one or more portions of the text with the set of images.

12. The method of claim 1, wherein generating the video surgical report comprises:
generating, using the one or more machine learning models, text for the at least some of the set of images; and
generating, using the one or more machine learning models, audio based on the text, the video surgical report comprising the audio.

13. The method of claim 1, further comprising:
receiving, using one or more audio sensors, audio captured during the surgical procedure, wherein the video surgical report comprises at least some of the audio corresponding to the at least some of the set of images.

14. The method of claim 1, wherein generating the video surgical report comprises:
obtaining pre-generated text associated with content depicted by the at least some of the set of images;
obtaining user-provided text of audio captured during the surgical procedure; and
generating text for the video surgical report based on the pre-generated text and the user-provided text.

15. The method of claim 1, wherein generating the video surgical report comprises:
generating audio based on data stored in an audio profile of a user, the data comprising at least one of a pitch, a timbre, a loudness, or a modulation associated with the user.

16. The method of claim 1, wherein obtaining the one or more images comprises:
accessing video captured during the surgical procedure; and
extracting at least one video snippet from the video based on the surgical procedure, wherein the one or more images comprise the at least one video snippet.

17. The method of claim 1, further comprising:
obtaining one or more additional images captured subsequent to the surgical procedure; and
generating an updated video surgical report comprising at least some of the one or more additional images.

18. The method of claim 1, wherein generating the video surgical report comprises:
adding one or more additional images to the video surgical report based on a similarity between content depicted by the one or more additional images and content depicted by at least one of the set of images, wherein the one or more additional images are captured prior to the surgical procedure.

19. A non-transitory computer-readable medium storing computer program instructions that, when executed by one or more processors, effectuate a method comprising:
obtaining one or more images of a surgical procedure;
determining, using one or more machine learning models, a set of images from the one or more images based on the surgical procedure; and
generating a video surgical report for the surgical procedure comprising at least some of the set of images.

20. A system comprising:
memory storing computer program instructions; and
one or more processors configured to execute the computer program instructions to cause the one or more processors to perform a method comprising:
obtaining one or more images of a surgical procedure;
determining, using one or more machine learning models, a set of images from the one or more images based on the surgical procedure; and
generating a video surgical report for the surgical procedure comprising at least some of the set of images.
